(19) United States
(12) Patent Application Publication
(10) Pub. No.: US 2022/0208305 A1
(43) Pub. Date: Jun. 30, 2022

(54) ARTIFICIAL INTELLIGENCE DRIVEN THERAPY CURATION AND PRIORITIZATION

(71) Applicant: Tempus Labs, Inc., Chicago, IL (US)

(72) Inventors: Martin Bontrager, Rochester, NY (US); Ashleigh McBratney, Northfield, IL (US); Emily Kudalkar, Chicago, IL (US); Daniel Ben-Isvy, Northbrook, IL (US); Robert Huether, Napervile, IL (US); Andrew Walker, Morton Grove, IL (US); Norah Jacob, Fort Lauderdale, FL (US)

(21) Appl. No.: 17/546,049

(22) Filed: Dec. 9, 2021

Related U.S. Application Data
(60) Provisional application No. 63/130,504, filed on Dec. 24, 2020.

(51) Int. Cl. G16B 30/10; G16B 40/20; G16B 40/30; G16H 20/40; G06N 3/12

(52) U.S. Cl. CPC G16B 30/10; G16B 40/20; G06N 3/123; G16H 20/40; G16B 40/30

(57) ABSTRACT

A method for associating published media with a subject includes receiving a cancerous biological specimen, sequencing it to obtain subject genomic data, identifying first and second alteration nomenclature matches to the subject genomic data in data extracted from first and second published media, applying a hierarchical rule set to the media based on the alteration nomenclature matches and one or more evidence metrics, the hierarchical rule set resulting in reporting a first treatment in the first medium and excluding reporting of a second treatment in the second medium despite a match between the second medium and subject disease states, identifying a reporting template based on the subject genomic data and the disease state, generating a report using the identified template, the report reporting treatments according to the hierarchical rule set, comparing the report to one or more approval criteria, and publishing the report when the approval criteria are satisfied.

### CROSS REFERENCE TO RELATED APPLICATIONS

This application claims the benefit of priority to U.S. provisional patent application No. 63/130,504, filed Dec. 24, 2020.

### BACKGROUND

Being able to identify therapies from the sea of publications, and provide the most relevant therapeutic, prognostic or diagnostic information for a selected patient, is not a simple or straightforward problem. There are millions of publications reporting on the results from testing potential therapies that might be relevant for patients. New publications are issued at a rate of about 9,000 publications each day. In a health system that addresses multiple disease states, publications should be reviewed and vetted to determine which publications provide relevant information for which patient populations and which disease states. There also is a need to identify the most therapeutically relevant articles—such as the top 3 to 6 articles—that may be provided to a physician for review, based on their patient's unique clinical and molecular makeup.

Publications may be manually curated to determine relevance. Manual curation, however, is an incredibly laborious and manual process that requires highly trained individuals to find relevant information and critically analyze scientific findings. There is a need for systems and methods that help to identify relevant literature, determine the key findings, and derive a summary of the pertinent information.

There also is a need, in the clinical laboratory industry, to determine which therapies should be provided on a clinical lab report in a manner that is specific to the patient's clinical and molecular makeup. Providing such relevant targeted therapies on a clinical lab report, such as a comprehensive genomic profile report, is incredibly manual. Current methods often do not allow for continuous update as new evidence is released. Current methods often do not include or take into consideration a listing of all sources of knowledge relevant to the patient's medical condition. Current methods also suffer from manual review which increases the period of time before which a physician is provided with the lab results. Current methods require time and effort from highly trained individuals that could be spent instead on analyzing new and/or improved actionable evidence. Current methods generally are not built in a framework that permits incorporation of new precision medicine datasets for lab reporting. There is a need for systems and methods that regularly update therapeutic recommendations on a highly specific, relevant, and patient-by-patient basis.

### SUMMARY

In one non-limiting aspect, the present disclosure provides a method for associating a published media with a subject. The method includes extracting genomic data, a disease state, a treatment, and an outcome from the published media, the genomic data including a pattern of gene expression and a genomic type, the treatment associated with an outcome when treating the disease state expressing the pattern of gene expression, identifying an alteration nomenclature match to the genomic data, scoring the treatment based at least in part on a similarity match to a disease state ontology and one or more evidence metrics, ranking each treatment for a disease state based at least in part on the treatment score to generate a group of high ranking treatments, and associating one or more the published media associated with the group of high ranking treatments with the subject when the subject is diagnosed with the disease state expressing the pattern of gene expression.

In the method, the published media can be selected from one of the following: written media, video media, audio, or audio/visual media.

In the method, the pattern of gene expression can be a sequence of nucleotides.

In the method, the pattern of gene expression can be an amino acid change.

In the method, the pattern of gene expression can be a nomenclature associated with a sequence of nucleotides.

In the method, the pattern of gene expression can be a gene symbol.

In the method, the pattern of gene expression can be a molecular biomarker.

In the method, the disease state can be cancer, cardiology, depression, mental health, diabetes, infectious disease, epilepsy, dermatology, or autoimmune disease.

In the method, a cancer treatment included in the treatment can be selected from surgery, chemotherapy, radiation therapy, bone marrow transplant, immunotherapy, hormone therapy, targeted drug therapy, cryoablation, radiofrequency ablation, a medication, or a clinical trial.

In the method, the genomic type can be a type of alteration. In some embodiments, the type of alteration can be a single-nucleotide polymorphism, multiple-nucleotide polymorphism, insertion, deletion, duplication, mutation, frame shift, repeat expansion, fusion, methylation, or copy number variation.

In the method, the genomic type can be a molecular function. In some embodiments, the molecular function can be a loss of function or a gain of function.

In the method, the genomic type can be a nucleotide location within a sequence of nucleotides.

In the method, the outcome can be a measurable change in health, function, or quality of life.

In the method, the outcome can be a prognosis or side effect.

In the method, the similarity match to the disease state ontology can include identifying the disease state within the ontology closest in semantic meaning to the disease state and assigning a score based at least in part on a difference in semantic meaning.

In the method, the similarity match to the disease state ontology can include a closest organ to the disease state.

In the method, the similarity match to the disease state ontology can include identifying a most similar disease state based at least in part on genomic similarities.

In the method, the similarity match to the disease state ontology can include identifying a most similar disease state based at least in part on a first cohort of patients having the disease state and a second cohort having a most similar disease state.

In the method, the alteration nomenclature can be HGVS.

In the method, the alteration nomenclature can be DNA alteration.

In the method, the alteration nomenclature can be RNA alteration.

In the method, the alteration nomenclature can be protein coding variant.

In the method, the alteration nomenclature can be MSI.

In the method, the alteration nomenclature can be HRD.

In the method, the alteration nomenclature can be upregulation of a gene pathway.

In the method, the alteration nomenclature can be downregulation of a gene pathway.

In the method, the alteration nomenclature can be presence of a protein.

In the method, alteration nomenclature can be absence of a protein.

In the method, the alteration nomenclature can be methylation.

In the method, the alteration nomenclature can be an epigenetic alteration.

In the method, the alteration nomenclature can be a chromosomal modification.

In the method, scoring the treatment can further include identifying a classification of disease states from a disease state ontology and measuring a distance between layers of the identified classification and the disease state.

In the method, scoring the treatment can further include identifying the treatment is FDA approved and available to the subject.

In the method, scoring the treatment can further include characterizing a level of evidence presented in the published media. In some embodiments, characterizing a level of evidence can further include identifying the treatment within the National Comprehensive Cancer Network. In some embodiments, characterizing a level of evidence can further include identifying the treatment has FDA approval. In some embodiments, characterizing a level of evidence can further include identifying the treatment as administered within a clinical trial having more than 1000 patients. In some embodiments, characterizing a level of evidence can further include identifying the treatment as administered within a clinical trial having fewer than 1000 patients.

In the method, the disease state expressing the pattern of gene expression can include identifying the pattern of gene expression in a sequencing report for the subject.

The method can further include reporting one or more associated published media having matching gene data to the subject's sequencing report.

In the method, the genomic data can further include one or more additional patterns of gene expression. In some embodiments, the one or more additional patterns of gene expression can include a sequence of nucleotides. In some embodiments, the one or more additional patterns of gene expression can include an amino acid change. In some embodiments, the one or more additional patterns of gene expression can include a nomenclature associated with a sequence of nucleotides. In some embodiments, the one or more additional patterns of gene expression can include a gene symbol. In some embodiments, the one or more additional patterns of gene expression can include a molecular biomarker.

### BRIEF DESCRIPTION OF THE SEVERAL VIEWS OF THE DRAWINGS

FIG. 1 illustrates a system for implementing an artificial intelligence driven therapy curation and prioritization engine according to an embodiment.

FIG. 2 illustrates a system for generating evidentiary based therapeutic annotations according to an embodiment.

FIG. 3a illustrates the first stage of a system for generating annotations in a structured format, the first stage identifying gene matches to disambiguated variants and mutations according to an embodiment.

FIG. 3b illustrates the second stage of a system for generating annotations in a structured format, the second stage identifying drugs, therapies, procedures, and/or diseases to disambiguated effects and outcomes according to an embodiment.

FIG. 4 illustrates an exemplary article having an abstract and body according to an embodiment.

FIG. 5 illustrates an exemplary complete annotation for scoring and prioritization according to an embodiment.

FIG. 6 illustrates a plurality of exemplary complete annotation for scoring and prioritization according to an embodiment.

FIG. 7 illustrates an exemplary article having evidence in an abstract and body, according to one embodiment.

FIG. 8 illustrates a plurality of exemplary complete annotation for scoring and prioritization according to an embodiment.

FIG. 9 illustrates an abstract that has both expression and copy number gain evidence for resistance to Cetuximab may be curated.

FIG. 10 illustrates a plurality of exemplary complete annotation for scoring and prioritization according to an embodiment.

FIG. 11 illustrates a rule-based selection for identifying which evidence should be stored in the internal database, according to an embodiment of the invention.

FIG. 12 illustrates a therapy template for a variant and disease state according to an embodiment.

FIG. 13 is a flow diagram of a process for receiving a request for annotated evidence.

FIG. 14 is a listing of tissue types, example drugs to include on a clinical report, evidence level associated with each respective drug, for the respective tissue type, and a corresponding therapy score, according to an embodiment.

FIG. 15 is a chart comparing the matching evidence between different external databases with an internal database of a laboratory.

FIG. 16 is a flow diagram of a method for generating a clinical report after curating features from one or more publications and/or from identifying features in one or more sources of clinical information;

FIG. 17 is a flow diagram of an alternative method for generating a clinical report bypassing feature curation; and

FIG. 18 is an illustration of a block diagram of an implementation of a computer system in which some implementations of the disclosure may operate.

### DETAILED DESCRIPTION

### Definitions

“Publication” or “article” means a text with information about a medical or scientific subject. Examples include, but are not limited to, abstracts, posters, pre-prints, papers, and the like.

“Disease state” means a state of disease, such as cancer, cardiology, depression, mental health, diabetes, infectious disease, epilepsy, dermatology, autoimmune diseases, or other diseases. A disease state may reflect the presence or absence of disease in a subject, and when present may further reflect the severity of the disease.

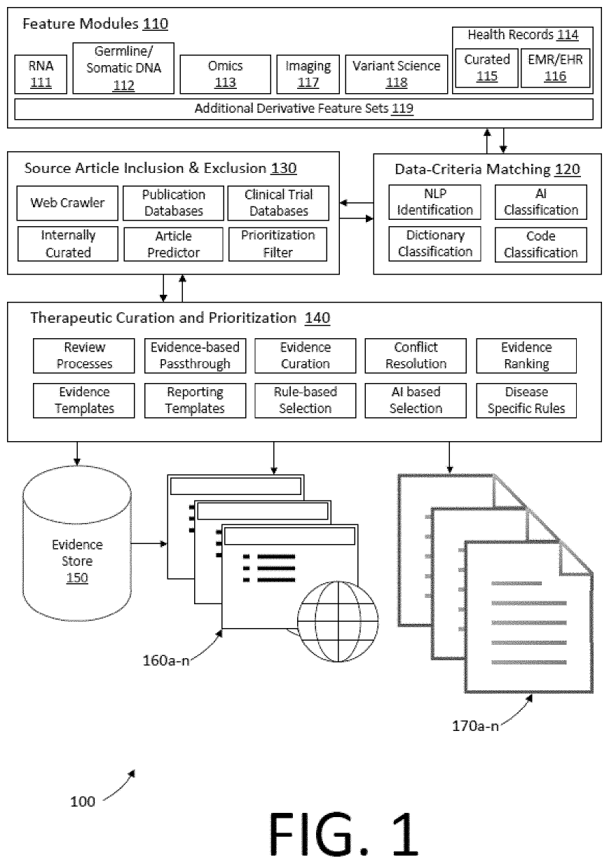

In this disclosure, a therapy curation and prioritization engine (or “therapy engine” for short) is disclosed. The therapy engine 100 may comprise features modules 110, data-criteria matching module 120, source article inclusion and exclusion module 130, therapeutic curation and prioritization module 140, an evidence store 150, a webform-based interactive user interface which, in some embodiments, may include webforms 160a-n, and electronic reports 170a-n may be generated and provided to the user via the graphical user interface (GUI). An example therapy engine 100 is shown in FIG. 1.

The feature modules 110 may store a collection of features, or status characteristics, generated for some or all patients whose information is present in the system 100. These features may be used to generate and model predictions using the system 100. While feature scope across all patients is informationally dense, a patient's feature set may be sparsely populated across the entirety of the collective feature scope of all features across all patients. For example, the feature scope across all patients may expand into the tens of thousands of features, while a patient's unique feature set may include a subset of hundreds or thousands of the collective feature scope based upon the records available for that patient. Each of these features may be used to identify one or more concepts within an article or publication and related to evidence that demonstrates the article or publication's importance to the patient based on the evidence extracted.

A plurality of features present in the feature modules 110 may include a diverse set of fields available within patient health records 114. Clinical information may be based upon fields which have been entered into an electronic medical record (EMR) or an electronic health record (EHR) 116, which can be done automatically or manually, e.g., by a physician, nurse, or other medical professional or representative. Other clinical information may be curated information (115) obtained from other sources, such as, for example, genetic sequencing reports (e.g., from molecular fields). Sequencing may include next-generation sequencing (NGS) and may be long-read, short-read, or other forms of sequencing a patient's somatic and/or normal genome. A comprehensive collection of features in additional feature modules may combine a variety of features together across varying fields of medicine which may include diagnoses, responses to treatment regimens, genetic profiles, clinical and phenotypic characteristics, and/or other medical, geographic, demographic, clinical, molecular, or genetic features. For example, as shown in FIG. 1, a subset of features may comprise molecular data features, such as features derived from an RNA feature module 111 or a DNA feature module 112 sequencing.

As further shown in FIG. 1, another subset of features, imaging features from imaging feature module 117, may comprise features identified through review of a specimen by pathologist, such as, e.g., a review of stained H&E or IHC slides. As another example, a subset of features may comprise derivative features obtained from the analysis of the individual and combined results of such feature sets. Features derived from DNA and RNA sequencing may include genetic variants from variant science module 118, which can be identified in a sequenced sample. Further analysis of the genetic variants present in variant science module 118 may include steps such as identifying single or multiple nucleotide polymorphisms, identifying whether a variation is an insertion or deletion event, identifying loss or gain of function, identifying fusions, calculating copy number variation, calculating microsatellite instability, calculating tumor mutational burden, or other structural variations within the DNA and RNA. Analysis of slides for H&E staining or IHC staining may reveal features such as tumor infiltration, programmed death-ligand 1 (PD-L1) status, human leukocyte antigen (HLA) status, or other immunology-related features.

Features derived from structured, curated, and/or electronic medical or health records 114 may include clinical features such as diagnosis, symptoms, therapies, outcomes, patient demographics such as patient name, date of birth, gender, ethnicity, date of death, address, smoking status, diagnosis dates for cancer, illness, disease, diabetes, depression, other physical or mental maladies, personal medical history, family medical history, clinical diagnoses such as date of initial diagnosis, date of metastatic diagnosis, cancer staging, tumor characterization, tissue of origin, treatments and outcomes such as line of therapy, therapy groups, clinical trials, medications prescribed or taken, surgeries, radiotherapy, imaging, adverse effects, associated outcomes, genetic testing and laboratory information such as performance scores, lab tests, pathology results, prognostic indicators, date of genetic testing, testing provider used, testing method used, such as genetic sequencing method or gene panel, gene results, such as included genes, variants, expression levels/statuses, or corresponding dates associated with any of the above.

As shown in FIG. 1, omics may be derived by Omics module 113 from information from additional medical or research based Omics fields including proteome, transcriptome, epigenome, metabolome, microbiome, and other multi-omic fields. Features derived from an organoid modeling lab may include the DNA and RNA sequencing information germane to each organoid and results from treatments applied to those organoids. Features 117 derived from imaging data may further include reports associated with a stained slide, size of tumor, tumor size differentials over time including treatments during the period of change, as well as machine learning approaches for classifying PDL1 status, HLA status, or other characteristics from imaging data. Other features may include additional derivative features sets 119 derived using other machine learning approaches based at least in part on combinations of any new features and/or those listed above. For example, imaging results may need to be combined with MSI calculations derived from RNA expressions to determine additional further imaging features. As another example, a machine learning model may generate a likelihood that a patient's cancer will metastasize to a particular organ or a patient's future probability of metastasis to yet another organ in the body. Other features that may be extracted from medical information may also be used. There are many thousands of features, and the above-described types of features are merely representative and should not be construed as a complete listing of features.

Additional derivative feature sets 119 may comprise stored alterations and stored classifications from a structural variant classification. An alteration module may be one or more microservices, servers, scripts, or other executable algorithms which generate alteration features associated with de-identified patient features from the feature collection. Exemplary alterations modules may include one or more of the following alterations as a collection of alteration modules. An SNP (single-nucleotide polymorphism) module may identify a substitution of a single nucleotide that occurs at a specific position in the genome, where each variation is present to some appreciable degree within a population (e.g. >1%). For example, at a specific base position, or loci, in the human genome, the C nucleotide may appear in most individuals, but in a minority of individuals, the position is occupied by an A. This means that there is a SNP at this specific position and the two possible nucleotide variations, C or A, are said to be alleles for this position. SNPs underline differences in susceptibility to a wide range of diseases (e.g. —sickle-cell anemia, β-thalassemia and cystic fibrosis result from SNPs).

The severity of illness and the way the body responds to treatments are also manifestations of genetic variations. For example, a single-base mutation in the APOE (apolipoprotein E) gene is associated with a lower risk for Alzheimer's disease. A single-nucleotide variant (SNV) is a variation in a single nucleotide without any limitations of frequency and may arise in somatic cells. A somatic single-nucleotide variation (e.g., caused by cancer) may also be called a single-nucleotide alteration. An MNP (Multiple-nucleotide polymorphisms) module may identify the substitution of consecutive nucleotides at a specific position in the genome. An InDels module may identify an insertion or deletion of bases in the genome of an organism classified among small genetic variations. While usually measuring from 1 to 10,000 base pairs in length, a microindel is defined as an indel that results in a net change of 1 to 50 nucleotides. Indels can be contrasted with a SNP or point mutation. An indel inserts and deletes nucleotides from a sequence, while a point mutation is a form of substitution that replaces one of the nucleotides without changing the overall number in the DNA. Indels, being either insertions, or deletions, can be used as genetic markers in natural populations, especially in phylogenetic studies. Indel frequency tends to be markedly lower than that of single nucleotide polymorphisms (SNP), except near highly repetitive regions, including homopolymers and microsatellites. An MSI (microsatellite instability) module may identify genetic hypermutability (predisposition to mutation) that results from impaired DNA mismatch repair (MMR). The presence of MSI represents phenotypic evidence that MMR is not functioning normally. MMR corrects errors that spontaneously occur during DNA replication, such as single base mismatches or short insertions and deletions. The proteins involved in MMR correct polymerase errors by forming a complex that binds to the mismatched section of DNA, excises the error, and inserts the correct sequence in its place. Cells with abnormally functioning MMR are unable to correct errors that occur during DNA replication and consequently accumulate errors. This causes the creation of novel microsatellite fragments. Polymerase chain reaction-based assays can reveal these novel microsatellites and provide evidence for the presence of MSI. Microsatellites are repeated sequences of DNA. These sequences can be made of repeating units of one to six base pairs in length. Although the length of these microsatellites is highly variable from person to person and contributes to the individual DNA “fingerprint”, each individual has microsatellites of a set length. The most common microsatellite in humans is a dinucleotide repeat of the nucleotides C and A, which occurs tens of thousands of times across the genome. Microsatellites are also known as simple sequence repeats (SSRs). A TMB (tumor mutational burden) module may identify a measurement of mutations carried by tumor cells and is a predictive biomarker being studied to evaluate its association with response to Immuno-Oncology (I-O) therapy. Tumor cells with high TMB may have more neoantigens, with an associated increase in cancer-fighting T cells in the tumor microenvironment and periphery. These neoantigens can be recognized by T cells, inciting an anti-tumor response. TMB has emerged more recently as a quantitative marker that can help predict potential responses to immunotherapies across different cancers, including melanoma, lung cancer and bladder cancer. TMB is defined as the total number of mutations per coding area of a tumor genome. Importantly, TMB is consistently reproducible. It provides a quantitative measure that can be used to better inform treatment decisions, such as selection of targeted or immunotherapies or enrollment in clinical trials. A CNV (copy number variation) module may identify deviations from the normal genome and any subsequent implications from analyzing genes, variants, alleles, or sequences of nucleotides. CNV are the phenomenon in which structural variations may occur in sections of nucleotides, or base pairs, that include repetitions, deletions, or inversions.

A Fusions module may identify hybrid genes formed from two previously separate genes. It can occur as a result of: translocation, interstitial deletion, or chromosomal inversion. Gene fusion plays an important role in tumorgenesis. Fusion genes can contribute to tumor formation because fusion genes can produce much more active abnormal protein than non-fusion genes. Often, fusion genes are oncogenes that cause cancer; these include BCR-ABL, TEL-AML1 (ALL with t(12; 21)), AML1-ETO (M2 AML with t(8; 21)), and TMPRSS2-ERG with an interstitial deletion on chromosome 21, often occurring in prostate cancer. In the case of TMPRSS2-ERG, by disrupting androgen receptor (AR) signaling and inhibiting AR expression by oncogenic ETS transcription factor, the fusion product regulates the prostate cancer. Most fusion genes are found from hematological cancers, sarcomas, and prostate cancer. BCAM-AKT2 is a fusion gene that is specific and unique to high-grade serous ovarian cancer. Oncogenic fusion genes may lead to a gene product with a new or different function from the two fusion partners. Alternatively, a proto-oncogene is fused to a strong promoter, and thereby the oncogenic function is set to function by an upregulation caused by the strong promoter of the upstream fusion partner. The latter is common in lymphomas, where oncogenes are juxtaposed to the promoters of the immunoglobulin genes. Oncogenic fusion transcripts may also be caused by trans-splicing or read-through events. Since chromosomal translocations play such a significant role in neoplasia, a specialized database of chromosomal aberrations and gene fusions in cancer has been created. This database is called Mitelman Database of Chromosome Aberrations and Gene Fusions in Cancer. An IHC (Immunohistochemistry) module may identify antigens (proteins) in cells of a tissue section by exploiting the principle of antibodies binding specifically to antigens in biological tissues. IHC staining is widely used in the diagnosis of abnormal cells such as those found in cancerous tumors. Specific molecular markers are characteristic of particular cellular events such as proliferation or cell death (apoptosis). IHC is also widely used in basic research to understand the distribution and localization of biomarkers and differentially expressed proteins in different parts of a biological tissue. Visualizing an antibody-antigen interaction can be accomplished in a number of ways. In the most common instance, an antibody is conjugated to an enzyme, such as peroxidase, that can catalyze a color-producing reaction in immunoperoxidase staining. Alternatively, the antibody can also be tagged to a fluorophore, such as fluorescein or rhodamine in immunofluorescence. Approximations from RNA expression data, H&E slide imaging data, or other data may be generated. For example, in some embodiments, the predictions may include PD-L1 prediction from H&E and/or RNA.

A Therapies module may identify differences in cancer cells (or other cells near them) that help them grow and thrive and drugs that “target” these differences. Treatment with these drugs is called targeted therapy. For example, many targeted drugs go after the cancer cells' inner ‘programming’ that makes them different from normal, healthy cells, while leaving most healthy cells alone. Targeted drugs may block or turn off chemical signals that tell the cancer cell to grow and divide; change proteins within the cancer cells so the cells die; stop making new blood vessels to feed the cancer cells; trigger your immune system to kill the cancer cells; or carry toxins to the cancer cells to kill them, but not normal cells. Some targeted drugs are more “targeted” than others. Some might target only a single change in cancer cells, while others can affect several different changes. Others boost the way your body fights the cancer cells. This can affect where these drugs work and what side effects they cause.

In some embodiments, matching targeted therapies may include identifying the therapy targets in the patients and satisfying any other inclusion or exclusion criteria. A VUS (variant of unknown significance) module may identify variants which are called but cannot be classified as pathogenic or benign at the time of calling. VUS may be catalogued from publications regarding a VUS to identify if they may be classified as benign or pathogenic. A Trial module may identify and test hypotheses for treating cancers having specific characteristics by matching features of a patient to clinical trials. These trials have inclusion and exclusion criteria that must be matched to enroll which may be ingested and structured from publications, trial reports, or other documentation. An Amplifications module may identify genes which increase in count disproportionately to other genes. Amplifications may cause a gene having the increased count to go dormant, become overactive, or operate in another unexpected fashion. Amplifications may be detected at a gene level, variant level, RNA transcript or expression level, or even a protein level. Detections may be performed across all the different detection mechanisms or levels and validated against one another. An Isoforms module may identify alternative splicing (AS), the biological process in which more than one mRNA (isoforms) is generated from the transcript of a same gene through different combinations of exons and introns. It is estimated by large-scale genomics studies that 30-60% of mammalian genes are alternatively spliced. The possible patterns of alternative splicing for a gene can be very complicated and the complexity increases rapidly as the number of introns in a gene increases. In silico alternative splicing prediction may find large insertions or deletions within a set of mRNA sharing a large portion of aligned sequences by identifying genomic loci through searches of mRNA sequences against genomic sequences, extracting sequences for genomic loci and extending the sequences at both ends up to 20 kb, searching the genomic sequences (repeat sequences have been masked), extracting splicing pairs (two boundaries of alignment gap with GT-AG consensus or with more than two expressed sequence tags aligned at both ends of the gap), assembling splicing pairs according to their coordinates, determining gene boundaries (splicing pair predictions are generated to this point), generating predicted gene structures by aligning mRNA sequences to genomic templates, and comparing splicing pair predictions and gene structure predictions to find alternative spliced isoforms. A Pathways module may identify defects in DNA repair pathways which enable cancer cells to accumulate genomic alterations that contribute to their aggressive phenotype. Cancerous tumors rely on residual DNA repair capacities to survive the damage induced by genotoxic stress which leads to isolated DNA repair pathways being inactivated in cancer cells. DNA repair pathways are generally thought of as mutually exclusive mechanistic units handling different types of lesions in distinct cell cycle phases. Recent preclinical studies, however, provide strong evidence that multifunctional DNA repair hubs, which are involved in multiple conventional DNA repair pathways, are frequently altered in cancer. Identifying pathways which may be affected may lead to important patient treatment considerations. A Raw Counts module may identify a count of the variants that are detected from the sequencing data. For DNA, this may be the number of reads from sequencing which correspond to a particular variant in a gene. For RNA, this may be the gene expression counts or the transcriptome counts from sequencing.

Structural variant classification may evaluate features herein, including alterations from alteration module, and other classifications from within itself from one or more classification modules. Structural variant classification may provide classifications to stored classifications for storage. An exemplary classification module may include a classification of a CNV as “Reportable” may mean that the CNV has been identified in one or more reference databases as influencing the tumor cancer characterization, disease state, or pharmacogenomics, “Not Reportable” may mean that the CNV has not been identified as such, and “Conflicting Evidence” may mean that the CNV has both evidence suggesting “Reportable” and “Not Reportable.” Furthermore, a classification of therapeutic relevance is similarly ascertained from any reference datasets mention of a therapy which may be impacted by the detection (or non-detection) of the CNV. Other classifications may include applications of machine learning algorithms, neural networks, regression techniques, graphing techniques, inductive reasoning approaches, or other artificial intelligence evaluations within modules. A classifier for clinical trials may include evaluation of variants identified from the alteration module which have been identified as significant or reportable, evaluation of all clinical trials available to identify inclusion and exclusion criteria, mapping the patient's variants and other information to the inclusion and exclusion criteria, and classifying clinical trials as applicable to the patient or as not applicable to the patient. Similar classifications may be performed for therapies, loss-of-function, gain-of-function, diagnosis, microsatellite instability, tumor mutational burden, indels, SNP, MNP, fusions, and other alterations which may be classified based upon the results of the alteration modules.

In addition to the above features and enumerated modules, the feature modules 110 may further include one or more of the modules that are described below and that can be included within respective modules of the Feature modules 110, as a sub-module or as a stand-alone module.

Continuing with FIG. 1, a germline/somatic DNA feature module 112 may comprise a feature collection associated with the DNA-derived information of a patient and/or a patient's tumor. These features may include raw sequencing results, such as those stored in FASTQ, BAM, VCF, or other sequencing file types known in the art; genes; mutations; variant calls; and variant characterizations. Genomic information from a patient's normal sample may be stored as germline and genomic information from a patient's tumor sample may be stored as somatic.

An RNA feature module 111 may comprise a feature collection associated with the RNA-derived information of a patient, such as transcriptome information. These features may include, for example, raw sequencing results, transcriptome expressions, genes, mutations, variant calls, and variant characterizations. Features may also include normalized sequencing results, such as those normalized by TMP.

The feature modules 110 can comprise various other modules. For example, a metadata module (not shown) may comprise a feature collection associated with the human genome, protein structures and their effects, such as changes in energy stability based on a protein structure.

A clinical module (not shown) may comprise a feature collection associated with information derived from clinical records of a patient, which can include records from family members of the patient. These may be abstracted from unstructured clinical documents, EMR, EHR, or other sources of patient history. Information may include patient symptoms, diagnosis, treatments, medications, therapies, hospice, responses to treatments, laboratory testing results, medical history, geographic locations of each, demographics, or other features of the patient which may be found in the patient's medical record. Information about treatments, medications, therapies, and the like may be ingested as a recommendation or prescription and/or as a confirmation that such treatments, medications, therapies, and the like were administered or taken.

An imaging module, such as, e.g., the imaging module 117, may comprise a feature collection associated with information derived from imaging records of a patient. Imaging records may include H&E slides, IHC slides, radiology images, and other medical imaging information, as well as related information from pathology and radiology reports, which may be ordered by a physician during the course of diagnosis and treatment of various illnesses and diseases. These features may include TMB, ploidy, purity, nuclear-cytoplasmic ratio, large nuclei, cell state alterations, biological pathway activations, hormone receptor alterations, immune cell infiltration, immune biomarkers of MMR, MSI, PDL1, CD3, FOXP3, HRD, PTEN, PIK3CA; collagen or stroma composition, appearance, density, or characteristics; tumor budding, size, aggressiveness, metastasis, immune state, chromatin morphology; and other characteristics of cells, tissues, or tumors for prognostic predictions.

An epigenome module, such as, e.g., an epigenome module from Omics module 113, may comprise a feature collection associated with information derived from DNA modifications which are not changes to the DNA sequence and regulate the gene expression. These modifications can be a result of environmental factors based on what the patient may breathe, eat, or drink. These features may include DNA methylation, hi stone modification, or other factors which deactivate a gene or cause alterations to gene function without altering the sequence of nucleotides in the gene.

A microbiome module, such as, e.g., a microbiome module from Omics module 113, may comprise a feature collection associated with information derived from the viruses and bacteria of a patient. These features may include viral infections which may affect treatment and diagnosis of certain illnesses as well as the bacteria present in the patient's gastrointestinal tract which may affect the efficacy of medicines ingested by the patient.

A proteome module, such as, e.g., a proteome module from Omics module 113, may comprise a feature collection associated with information derived from the proteins produced in the patient. These features may include protein composition, structure, and activity; when and where proteins are expressed; rates of protein production, degradation, and steady-state abundance; how proteins are modified, for example, post-translational modifications such as phosphorylation; the movement of proteins between subcellular compartments; the involvement of proteins in metabolic pathways; how proteins interact with one another; or modifications to the protein after translation from the RNA such as phosphorylation, ubiquitination, methylation, acetylation, glycosylation, oxidation, or nitrosylation.

Additional Omics module(s) (not shown) may also be included in Omics module 113, such as a feature collection associated with all the different field of omics, including: cognitive genomics, a collection of features comprising the study of the changes in cognitive processes associated with genetic profiles; comparative genomics, a collection of features comprising the study of the relationship of genome structure and function across different biological species or strains; functional genomics, a collection of features comprising the study of gene and protein functions and interactions including transcriptomics; interactomics, a collection of features comprising the study relating to large-scale analyses of gene-gene, protein-protein, or protein-ligand interactions; metagenomics, a collection of features comprising the study of metagenomes such as genetic material recovered directly from environmental samples; neurogenomics, a collection of features comprising the study of genetic influences on the development and function of the nervous system; pangenomics, a collection of features comprising the study of the entire collection of gene families found within a given species; personal genomics, a collection of features comprising the study of genomics concerned with the sequencing and analysis of the genome of an individual such that once the genotypes are known, the individual's genotype can be compared with the published literature to determine likelihood of trait expression and disease risk to enhance personalized medicine suggestions; epigenomics, a collection of features comprising the study of supporting the structure of genome, including protein and RNA binders, alternative DNA structures, and chemical modifications on DNA; nucleomics, a collection of features comprising the study of the complete set of genomic components which form the cell nucleus as a complex, dynamic biological system; lipidomics, a collection of features comprising the study of cellular lipids, including the modifications made to any particular set of lipids produced by a patient; proteomics, a collection of features comprising the study of proteins, including the modifications made to any particular set of proteins produced by a patient; immunoproteomics, a collection of features comprising the study of large sets of proteins involved in the immune response; nutriproteomics, a collection of features comprising the study of identifying molecular targets of nutritive and non-nutritive components of the diet including the use of proteomics mass spectrometry data for protein expression studies; proteogenomics, a collection of features comprising the study of biological research at the intersection of proteomics and genomics including data which identifies gene annotations; structural genomics, a collection of features comprising the study of 3-dimensional structure of every protein encoded by a given genome using a combination of modeling approaches; glycomics, a collection of features comprising the study of sugars and carbohydrates and their effects in the patient; foodomics, a collection of features comprising the study of the intersection between the food and nutrition domains through the application and integration of technologies to improve consumer's well-being, health, and knowledge; transcriptomics, a collection of features comprising the study of RNA molecules, including mRNA, rRNA, tRNA, and other non-coding RNA, produced in cells; metabolomics, a collection of features comprising the study of chemical processes involving metabolites, or unique chemical fingerprints that specific cellular processes leave behind, and their small-molecule metabolite profiles; metabonomics, a collection of features comprising the study of the quantitative measurement of the dynamic multiparametric metabolic response of cells to pathophysiological stimuli or genetic modification; nutrigenetics, a collection of features comprising the study of genetic variations on the interaction between diet and health with implications to susceptible subgroups; cognitive genomics, a collection of features comprising the study of the changes in cognitive processes associated with genetic profiles; pharmacogenomics, a collection of features comprising the study of the effect of the sum of variations within the human genome on drugs; pharmacomicrobiomics, a collection of features comprising the study of the effect of variations within the human microbiome on drugs; toxicogenomics, a collection of features comprising the study of gene and protein activity within particular cell or tissue of an organism in response to toxic substances; mitointeractome, a collection of features comprising the study of the process by which the mitochondria proteins interact; psychogenomics, a collection of features comprising the study of the process of applying the powerful tools of genomics and proteomics to achieve a better understanding of the biological substrates of normal behavior and of diseases of the brain that manifest themselves as behavioral abnormalities, including applying psychogenomics to the study of drug addiction to develop more effective treatments for these disorders as well as objective diagnostic tools, preventive measures, and cures; stem cell genomics, a collection of features comprising the study of stem cell biology to establish stem cells as a model system for understanding human biology and disease states; connectomics, a collection of features comprising the study of the neural connections in the brain; microbiomics, a collection of features comprising the study of the genomes of the communities of microorganisms that live in the digestive tract; cellomics, a collection of features comprising the study of the quantitative cell analysis and study using bioimaging methods and bioinformatics; tomomics, a collection of features comprising the study of tomography and omics methods to understand tissue or cell biochemistry at high spatial resolution from imaging mass spectrometry data; ethomics, a collection of features comprising the study of high-throughput machine measurement of patient behavior; and videomics, a collection of features comprising the study of a video analysis paradigm inspired by genomics principles, where a continuous digital image sequence, or a video, can be interpreted as the capture of a single image evolving through time of mutations revealing patient insights.

In some embodiments, a robust collection of features may include all of the features disclosed above. However, predictions based on the available features may include models which are optimized and trained from a selection of fewer features than in an exhaustive feature set. Such a constrained feature set may include, in some embodiments, from tens to hundreds of features. For example, a prediction may include predicting the likelihood a patient's tumor may metastasize to the brain. A model's constrained feature set may include the genomic results of a sequencing of the patient's tumor, derivative features based upon the genomic results, the patient's tumor origin, the patient's age at diagnosis, the patient's gender and race, and symptoms that the patient brought to their physicians attention during a routine checkup.

Data-Criteria Matching 120 interfaces with feature modules 110 and source article inclusion and exclusion 130 to use natural language processing (NLP) techniques for identifying key terms of an article or publication which match to a feature of feature module 110. Once a concept is extracted, the concept may be classified or mapped to a respective feature by a dictionary mapping, looking up a code classification, or through the use of artificial intelligence trained to classify the concept as a feature. Methods and techniques for the use of NLP to extract concepts from text and classify them as a feature are described in U.S. patent application Ser. No. 16/702,510, titled “Clinical Concept Identification, Extraction, And Prediction System And Related Methods”, and filed Dec. 3, 2019; and U.S. patent application Ser. No. 16/289,027, titled “Mobile Supplementation, Extraction, And Analysis Of Health Records”, and filed Feb. 28, 2019, both of which are incorporated by reference for all purposes herein.

Classification Codes for Mapping Features Between Data Stores

One embodiment of the feature to NLP extracted concept matching may assign classification codes to each feature of the patient data store and the corresponding concept. For example, a diagnosis of breast cancer may have a classification table, as shown, in part:

| Diagnosis | Code |
|---|---|
| Breast Cancer | 63050 |
| Ductal Carcinoma In Situ | 63051 |
| Invasive Ductal Carcinoma of the Breast | 63052 |
| Tubular Carcinoma of the Breast | 63053 |
| Medullary Carcinoma of the Breast | 63054 |
| Mucinous Carcinoma of the Breast | 63055 |
| Papillary Carcinoma of the Breast | 63056 |
| Cribriform Carcinoma of the Breast | 63057 |
| Invasive Lobular Carcinoma of the Breast | 63058 |

A treatment involving medications may have a classification table prioritized from brand names, chemical names, or other groupings, as shown, in part:

| Brand (Chemical) | Code |
|---|---|
| Abraxane (albumin-bound or nab-paclitaxel) | 77121 |
| Adriamycin (doxorubicin) | 77131 |

| Chemical (Brand) | Code |
|---|---|
| Carboplatin (Paraplatin) | 78141 |
| Daunorubicin (Cerubidine, DaunoXome) | 78151 |

DNA/RNA Molecular features may have a classification table for genetic mutations, variants, transcriptomes, cell lines, methods of evaluating expression (TPM, FPKM), the lab which provided the results:

| RNA | Code |
|---|---|
| OR6C69P - Overexpressed | 1013057 |
| OR6C69P - Normal | 1013058 |
| LINC02355 - Tempus Overexpressed | 1014028 |
| LINC02355 - Foundation Overexpressed | 1014029 |
| RPS4XP15 | 1015010 |

A data structure may relate the structured information as a classification code with the absolute value of the report result:

| Code | Value | |
|---|---|---|
| 1015010 | 85 | TPM |
| 1015010 | 20 | FPKM |

Features may be mapped according to the same classification conventions above, however, nested criteria or more complicated criteria may be converted to another format, such as JavaScript Object Notation (JSON) to preserve the inclusion or exclusion criteria in the proper format without any information loss.

For example, for features from a clinical trial, an inclusion criterion “Histologically or cytologically confirmed diagnosis of locally advanced or metastatic solid tumor that harbors an NTRK1/2/3, ROS1, or ALK gene rearrangement” may touch upon the following classification codes:

| Feature | Code |
|---|---|
| Histologically confirmed diagnosis | 20253 |
| Cytologically confirmed diagnosis | 20254 |
| Locally advanced | 20317 |
| Metastatic | 20439 |
| Solid tumor | 19001 |
| NTRK1 | 1013120 |
| NTRK2 | 1013121 |
| NTRK3 | 1013122 |
| ROS1 | 1013261 |
| ALK | 1013273 |

The inclusion criteria may be structured to represent: 19001 AND (20253 OR 20254) AND (20317 OR 20439) AND (1013120 OR 1013121 OR 1013122 OR 1013261 OR 1013273)

An inclusion criterion “At least 4 weeks must have elapsed since completion of antibody-directed therapy” may touch upon the following classification codes in a reduced-exemplary reference set:

| Feature | Code |
|---|---|
| Antibody Directed Therapy | 25001 |
| Monoclonal Antibody Therapy | 27015 |
| Nivolumab | 77233 |
| Avelumab | 77238 |
| Emapalumab | 77245 |
| Polyclonal Antibody Therapy | 27023 |
| . . . | |
| Hyperimmune Antibody Therapy | 27031 |
| . . . | |

In a first example, the inclusion criteria may be structured to represent: 25001 AND (Date Administered is Older than XX/YY/ZZZZ), where all therapies which fall under Antibody Directed Therapy are assigned multiple codes, a first code 25001 for antibody directed therapy; a second code 27015, 27023, or 27031 for the type of antibody therapy, and a third code 77233, 77238, 77245 for the specific medication applied as part of the antibody therapy. In another example, the structured inclusion criteria may list all of the therapy codes which qualify in addition to 25001.

Dictionary Classification for Mapping Between Data Stores

A second embodiment of the data store to inclusion/exclusion criteria (data-criteria) concept matching may utilize dictionary classification to each feature of the patient data store and the corresponding inclusion/exclusion criteria to identify relationships within the data that may not be immediately obvious. The process of enumerating known drugs into a list may include identifying clinical drugs prescribed by healthcare providers, pharmaceutical companies, and research institutions. Such providers, companies, and institutions may provide reference lists of their drugs. For example, the US National Library of Medicine (NLM) publishes a Unified Medical Language System (UMLS) including a Metathesaurus having drug vocabularies including CPT®, ICD-10-CM, LOINC®, MeSH®, RxNorm, and SNOMED CT®. Each of these drug vocabularies highlights and enumerates specific collections of relevant drugs. Other institutions such as insurance companies may also publish clinical drug lists providing all drugs covered by their insurance plans. By aggregating the drug listings from each of these providers, companies, and institutions, an enumerated list of clinical drugs that is universal in nature may be generated.

For example, “Tylenol” and “Tylenol 50 mg” may match in the dictionary from UMLS with a concept for “acetaminophen”. It may be necessary to explore the relationships between the identified concept from the UMLS dictionary and any other concepts of related dictionaries or the above universal dictionary. Though visualization is not required, these relationships may be visualized through a graph-based logic for following links between concepts that each specific integrated dictionary may provide.

The classification system may be applied to curate features and concepts extracted from text using a well-defined clinical/ontological dictionary to provide classifications based upon language concepts rather than codes.

Another embodiment may combine the code classification system with the dictionary classification system to use concept-based classification to an internal code index.

Artificial Intelligence for Predicting Patient Eligibility for Clinical Trials or Criteria

A third data-criteria concept mapping classification system may reside entirely within AI.

A machine learning algorithm (MLA) or a neural network (NN) may be trained from a training data set. For a data-criteria concept mapping classifier, an exemplary training data set may include patient information from the patient data store, clinical trial information including inclusion and exclusion criteria, and resulting line-by-line classification results for whether the inclusion or exclusion criteria were met.

MLAs include supervised algorithms (such as algorithms where the features/classifications in the data set are annotated) using linear regression, logistic regression, decision trees, classification and regression trees, Naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

NNs include conditional random fields, convolutional neural networks, attention based neural networks, long short term memory networks, or other neural models where the training data set includes a plurality of tumor samples, RNA expression data for each sample, and pathology reports covering imaging data for each sample. While MLA and neural networks identify distinct approaches to machine learning, the terms may be used interchangeably herein. Thus, a mention of MLA may include a corresponding NN or a mention of NN may include a corresponding MLA unless explicitly stated otherwise. Artificial NNs are efficient computing models which have shown their strengths in solving hard problems in artificial intelligence. They have also been shown to be universal approximators (can represent a wide variety of functions when given appropriate parameters). One of the major criticisms for NNs, is their being black boxes, since satisfactory explanation of their behavior may be difficult to discern. While research is ongoing to pierce the veil of NN learning, the rules driving the classification process are usually, and may continue to be, indecipherable black boxes. Similar constraints exist for some, but not all MLA. For example, some MLA may identify features of importance and identify a coefficient, or weight, to them. The coefficient may be multiplied with the occurrence frequency of the feature to generate a score, and once the scores of one or more features exceed a threshold, certain classifications may be predicted by the MLA. A coefficient schema may be combined with a rule-based schema to generate more complicated predictions, such as predictions based upon multiple features. For example, ten key features may be identified across three different classifications. A list of coefficients may exist for the features, and a rule set may exist for the classification. A rule set may be based upon the number of occurrences of the feature, the scaled weights of the features, or other qualitative and quantitative assessments of features encoded in logic known to those of ordinary skill in the art. In other MLA, features may be organized in a binary tree structure. For example, key features which distinguish between the most classifications may exist as the root of the binary tree and each subsequent branch in the tree until a classification may be awarded based upon reaching a terminal node of the tree. For example, a binary tree may have a root node which tests for a first feature. The occurrence or non-occurrence of this feature must exist (the binary decision), and the logic may traverse the branch which is true for the item being classified. Additional rules may be based upon thresholds, ranges, or other qualitative and quantitative tests.

While supervised methods are useful when the training dataset has many known values or annotations, the nature of EMR/EHR documents is that there may not be many annotations provided. When exploring large amounts of unlabeled data, unsupervised methods are useful for binning/bucketing instances in the data set. Returning to the example regarding gender, an unsupervised approach may attempt to identify a natural divide of documents into two groups without explicitly taking gender into account. On the other hand, a drawback to a purely unsupervised approach is that there's no guarantee that the division identified is related to gender. For example, the division may be between patients who went to a specific hospital and those who did not rather than the desired division.

Source Article inclusion and exclusion 130 comprises a number of article, publication, and other media searching tools, such as a web crawler, databases for storing publications, clinical trial databases, or even internally curated datasets which include references to one or more articles or publications as well as an article predictor, which will receive the curated and structured annotations from the article and predict the relationships from the differing ideas of the article to the matched data-criteria from module 120 and a prioritization filter which may identify the most relevant articles which should be added to the system 100 first and which articles may be low priority and can wait. Media may be one or more of written media, video media, audio media, or audio/visual media, including, e.g., publications, periodicals, articles, journals, reports, clinical trials, abstracts, studies, guidelines, books, film, video, images, lectures, webcasts, podcasts, conferences, notes, or reviews.

There are many sources which include relevant, therapeutically actionable articles and publications. For example, PubMed, Science Direct, Google Scholar, and other online sources may include extensive collections of articles. In another example, the FDA requires clinical trials to register before they may enroll patients and be held. These registered clinical trials may be referenced using a website, such as clinicaltrials.gov, which contains a complete listing of all clinical trials registered with the FDA. In addition to clinicaltrials.gov, other government-sponsored websites and private websites may exist for searching through clinical trials. A web crawler may periodically crawl these websites collecting detailed information from each article and add the collected evidentiary/therapeutically actionable information to an internally curated data storage. Institutions may also publish research papers identifying the purpose of a drug, treatment, or procedure as well as any information on the expected outcomes and effects of them. As new publications are published, they may be curated and the information added to the data storage. Curation may be performed by a medical professional, by a well-trained machine learning model, or a combination of both. Pharmaceutical companies or other institutions may maintain their own publicly available databases which may be queried to retrieve information. A periodic query may be sent to collect information and add it to the data storage. Each website, publication source, or database may be treated as an independent source of information. In another example, pharma-sponsored clinical trial protocols may provide detailed, dozens to hundreds of pages in reports on the detailed specifics of a clinical trial. Relationships forged between a pharmaceutical company and another partner for aggregating clinical trial information may include release of these protocols for deep learning purposes. These independent sources may be compared to one another for accuracy as a whole or aggregated across each collection medium (website, publication, database, protocols), where discrepancies between sources may be evaluated by a medical professional and/or deference given to the most respected source (as a whole or in each collection medium). Articles and/or publications may be routinely gathered via any of the collection mediums to identify new evidence or modifications to existing evidence which should be considered by a physician to effectively treat a patient. New evidence may be added to the data storage and any modifications may be updated to be reflected in the data storage. Continuing from the above example, detailed clinical trial information may include inclusion and exclusion criteria corresponding to any of the features stored in the comprehensive patient data store. Additional clinical trial information may include the study type (interventional/observational), study results, recruitment stage (not yet recruiting, recruiting, enrollment by invitation, suspended, unknown), title, planned measurement such as one described in the protocol that is used to determine the effect of an intervention/treatment on participants, interventions including drugs, medical devices, procedures, vaccines, and other products that are either investigational or already available, interventions including noninvasive approaches of education or modifying diet and exercise, sponsors or funders, geographic location (country, state, city, facility), trial stage such as those based on definitions developed by the FDA for the study's objective, the number of participants, and other characteristics (Early Phase 1, Phase 1, Phase 2, Phase 3, and Phase 4), or notable dates such as start and end dates. As each of these criteria are curated from their respective sources, a unified, internally-curated, and structured database may be formed to hold the criteria in the appropriate format for data-criteria concept matching.

Features in the patient data store may be aggregated from many different sources, each source potentially having their own organizational and identification schema for structuring the features within the source. One embodiment of the instant invention may convert all incoming features to a common, structured format of the patient data store. Similarly, evidentiary information may be aggregated from many different sources, each potentially having their own organizational and identification schema for structuring the clinical trial information within the source. One embodiment of the instant invention may also convert all incoming evidentiary information to the common, structured format of the patient data store as well as an intermediate concept mapping to preserve evidence of therapeutic effect, including inclusion and exclusion criteria in the original clinical trial information to match with the outcomes of a clinical trial.

Therapeutic curation and prioritization module 140 receives articles from source article inclusion and exclusion 130 for generation of structured, annotated evidence, module 140 comprises one or more manual or automated review processes, once evidence is generated, an automatic evidence-based passthrough may initiate, passing evidence to a report or to storage once specific criteria are met, an evidence curation module for removing redundant information from the evidence store, for example, if the evidence is already known, a conflict resolution module for resolving conflicts from two or more articles where evidence contradicts what is generally know, already stored in the evidence store 150, or new evidence that contradict each other, evidence template module for storing and evidence may be filled out according to an evidence template, reporting information may be generated based on the evidence or article surrounding the evidence for sharing the information with a physician, a rule based evidence selection module, an AI based evidence selection module, and a disease specific rule module. Modules within Therapeutic curation and prioritization 140 operate together to generate evidence annotations, qualify the evidence based upon the therapeutic impact a physician may need to be aware of, and add the information to reporting queues where one or more reports reference the genes, variants, drugs, therapies, or procedures for which the evidence supports actionable knowledge. Evidence may be ranked, or scored, to reflect the actionability of the evidence.

In an embodiment, the therapy prioritization engine can support highly specific therapy suggestions. The therapy prioritization engine may be based on evidence in a knowledge database, such as the evidence store 150, which may include references that have been flagged and added. The therapy engine 100 may permit therapeutic recommendations to be made on a patient-by-patient basis. The therapy engine 100 can account for the newest evidence, tissue and variant specific recommendations, as well as the presence of interacting variants.

Evidence store 150 may receive curated structured annotations generated from therapeutic curation and prioritization module 140 and store them for use in the system 100. Evidence may be stored in a structured format for retrieval by a user interface such as, for example, a webform-based interactive user interface which, in some embodiments, may include webforms 160a-n. Webforms may support GUIs that can be displayed by a computer to a user of the computer system for performing a plurality of analytical functions, including initiating or viewing the instant evidence. Electronic reports 170a-n may be generated and provided to the user via the graphical user interface (GUI). It should be appreciated that the GUI may be presented on a user device which is connected to a content server having therapy engine 100 via a network.

The reports 170a-n can be provided to the user as part of a network-based evidence management system that collects, converts and consolidates therapeutic information from various source articles into a standardized format, stores it in network-based storage devices, and generates messages comprising electronic reports once the reports are generated in accordance with embodiments of the present disclosure. For example, a report may provide sequencing results, pathogenic variants, and implicated therapies for review by a primary care physician, authorized medical professional, or patient. In this way, a user (e.g., a physician, oncologist, or any other health care provider, or a patient, receives computer-generated evidence relating to one or more disease states.

In one aspect, the language processing engine, such as the NLP identification within data-criteria matching 120, may comprise a support vector algorithm. The support vector algorithm may be implemented, for example, as a machine learning algorithm. The support vector algorithm may identify new publications of interest and may further assign each new publication a publication score, such as from 0-1, based on how likely the article belongs in the evidence knowledge database. In one aspect, the support vector algorithm may generate two scores of interest: how similar the new publication is to some or all other publications in the knowledge database and how similar the new publication is to other articles in the knowledge database that have been designated as high-quality therapeutic articles.

Subsequent to the application of the machine learning algorithm, the language processing engine may apply rule-based language (RBL) and a secondary ML engine to enrich for publications of interest and to provide annotation to help guide variant scientists in identifying articles of interest

Publications may be annotated via the RBL engine in terms of the (1) Genes, (2) Mutations, (3) Diseases, (4) Drugs, and (5) Therapeutic Effect to which the publication refers. These annotations, and the original ML scores, are then fed into the secondary ML algorithm and articles re-scored in terms of their expected value to the INTERNAL DATABASE. Annotations and scores are then stored and indexed so that users at Tempus can retrieve, for example, expected highly relevant articles about the gene EGFR in Lung Cancer and review those articles for inclusion in the INTERNAL DATABASE.

The language processing engine may be used to prioritize and pre-annotate publications, in order to identify therapeutic, prognostic, and diagnostic evidence to return on patient reports. The language processing engine may be used to significantly reduce the number of publications that must be analyzed by a person, such as a variant scientist, and allows for time to be spent curating relevant literature, rather than sifting through thousands of articles that may be irrelevant for patient care. In one example, articles may be bucketed based on the range of their score, such that scores exceeding a relevance threshold are shown to a evidentiary review process first, scores between the relevance threshold and a lower, no relevance threshold, are shown to an evidentiary review second, and then scores below the no relevance threshold are effectively hidden from the evidentiary review unless manual curation requests the evidence for review.

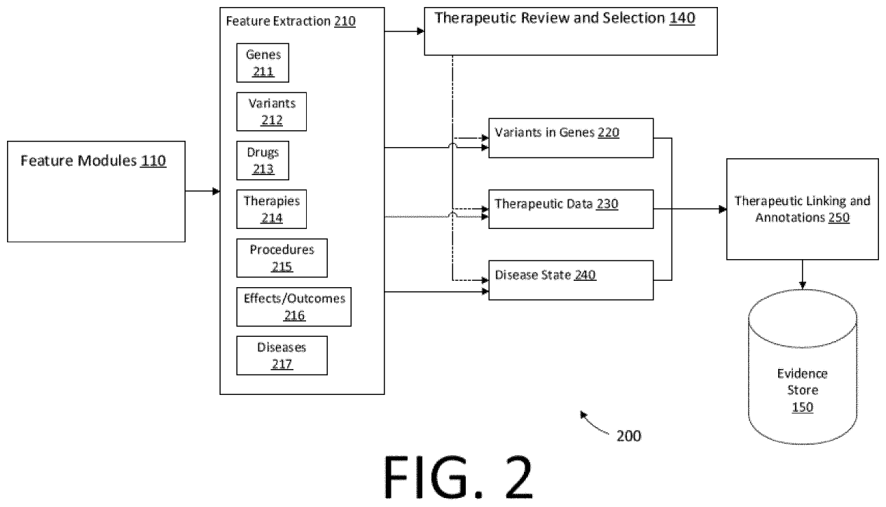

FIG. 2 illustrates a system for generating evidentiary based therapeutic annotations according to an embodiment. In one embodiment, a schematic of the metadata extracted by the therapy engine may include extracting a plurality of components from the abstract, or body, of the article or publication.

Feature extraction 210 may receive a listing of features from the feature modules 110 for which an article or publication may be scanned to identify linked evidentiary knowledge. In one example, evidentiary knowledge may include features such as one or more genes 211 or gene variants 212. An unidentified gene may be identified, for example, by extracting all presumed gene references from the abstract or body of an article or publication and comparing those genes to genes within the feature module 110. In one example, once each gene is verified as matching a gene from the genes within feature module 110, it may be appended to a gene list for evidentiary considerations. Similar matching may be performed, for example, with variants 212, drugs 213, therapies 214, procedures 215, effects and outcomes 216, and diseases 217. An orchestrator, such as the therapeutic review and selection module 140 may direct the matching of variants to genes, drugs, therapies, and/or procedures to their effects and/or outcomes, and diseases to their closest disease states for the therapeutic linking and annotations process 250. Evidence may then be stored in evidence store 150 for ranking, additional considerations, or review.

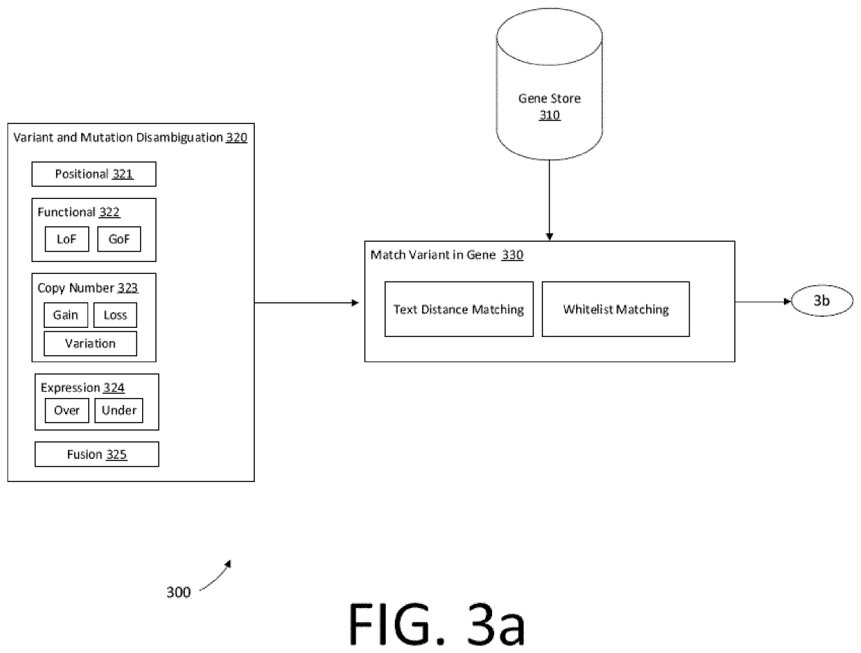

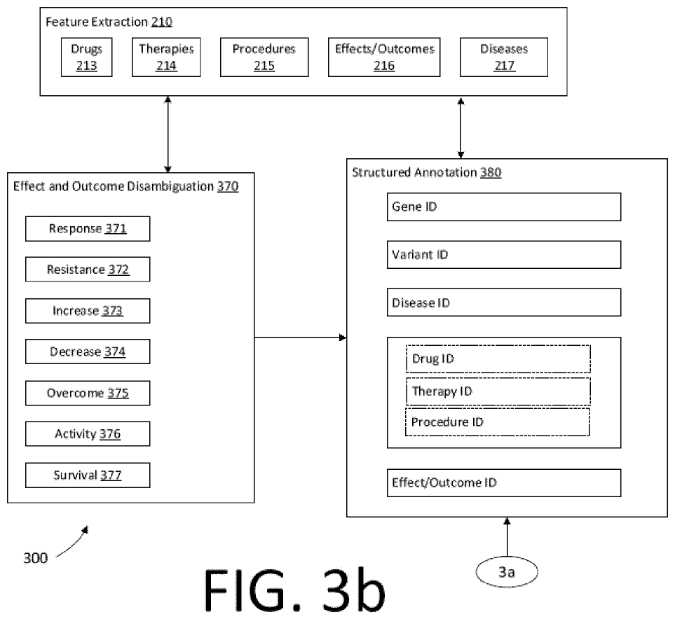

FIGS. 3a and 3b illustrate stages of generating annotations for evidence extracted from articles in a structured format. The first stage of a system for generating annotations in a structured format includes identifying gene matches to disambiguated variants and mutations and the second stage includes identifying drugs, therapies, procedures, and/or diseases to disambiguated effects and outcomes according to an embodiment.

Gene store 310 may be a redefined whitelist of genes for which new evidence may be curated or may be an exhaustive list of genes found within feature module 110. Variant and mutation disambiguation 320 may identify a specific classification of variant or mutation from the article and place it within a classification, or category, based on the type of variation as appeared, for example, as an SNP, MNP, InDel, etc., or may place it based on the type of function it accomplishes such as a positional variation 321, a functional variation 322 including a loss of function (LoF) or gain of function (GoF), a copy number alteration 323 of a resulting sequencing include a copy number gain, a copy number loss, or a copy number variation, an expression level 324 of a resulting sequencing including overexpression and underexpression, and a fusion event 325 including identifying a hybrid gene formed from two previously independent genes as a result of translocation, interstitial deletion, or chromosomal inversion. Each type may be associated with a different searching mechanism to identify and confirm a match between the variation and the gene. In one example, all variations may be listed in a whitelist having a corresponding gene which may be referenced. In another example, a positional variation or mutation may be referenced against each gene from gene store 310 to link variant to gene at stage 330 by text distance association or through a whitelist. In other examples, functional variations, copy number variations, expression levels, and fusions may directly map to a known variation when the variant is known and the evidence is to link the variant to a functional effect. Once matched, gene and variant may be provided to the second stage, such as depicted in FIG. 3b.

Feature extraction 210 may provide the matched drugs 213, therapies 214, procedures 215 and their matched effects/outcomes 216 to effect and outcome disambiguation 370 for classification to the structured format of the evidence store. In one example, this may include classifying each variant to one or more variant and/or mutation types. In one example, genetic variants may be structured into one or more of the following mutation types: Positional, Functional (GOF/LOF), Copy number variation (copy number gain/loss), Expression (Over-/Under-), Fusion. Each mutation type may be assigned based on searching for a set of terms. The regular expressions under each term define how a variant may be identified in one embodiment of a NLP model. The variant and mutation type may be described as the ‘variant annotations’. If a positional/exact variant is identified in the abstract, or body, that is put into the annotation. However, if no positional variants are found, the mechanism for the gene (GOF or LOF) may be used instead. Gene mechanisms for exemplary panels may be pre-curated and stored in a database or feature module 110.

Some positional variations may be matched according to a regular expression. Certain regular expressions may include a “?” operator that indicates either zero or one of the preceding token (e.g., a space, a character, and/or a minus sign). In one example, Positional variations may be matched according to a regular expression ‘[a−z]\d+[a−z]’ which may resolve a gene name to, for example, L858R, a regular expression ‘[a−z]\d+_[a−z]\d+delins[a−z]+’ which may resolve a gene name to, for example, S2215_L2216delinsF, a regular expression ‘\d+[atgc]?>?[atgc]’, which may resolve a gene name to, for example, 1900T>C, or a regular expression ‘exo?n?s? ?[\d−]+skipping’, which may resolve a gene name to, for example, exon 14 skipping or ex14 skipping. Gain of functions may be matched according to regular expression to match ‘gof’, ‘gain-? ?of-? ?function’, or ‘constitutiv\w+activ\w+’ which may resolve to, for example gof, gain-of-function, or constitutively active. Loss of function variations may be matched according to regular expressions ‘lof, loss-? ?of-? ?function’, or ‘inactivat\w+’ which may resolve to, for example, lof, loss-of-function, or inactivated. A copy number gain variation may be matched according to a regular expression ‘cng’, ‘copy [number]* ?gain’, or ‘cn ?>?\d+’, which may resolve to, for example, cng, copy number gain, copy gain, or CN>4. A copy number loss variation may be matched according to a regular expression ‘cnl’, ‘copy [number]* ?loss’, or ‘cn ?<?\d+’, which may resolve to, for example, cnl, copy number loss, copy loss, or CN<2. A copy number variation may be matched according to a regular expression ‘cnv’, ‘copy [number]* ?varian?t\w*’, which may resolve to cnv, copy number variant, or copy variation. Overexpression may be matched according to a regular expression ‘over-? ?express\w*’ or ‘high\w* express\w*’, which may resolve to, for example, overexpressing, over-expression or higher expression. Underexpression may be matched according to a regular expression ‘under? ?express\w*’ or ‘loss of [w−]* ? express\w+’, which mat resolve to, for example, underexpressing, under-expression, or loss of TP53 expression. General expression may be matched according to regular expression ‘express\w*’, which may resolve to, for example, expression. In an instance where expression is searched, it shall be searched after under and over expressions have been searched so that multiple matching terms may be excluded. A fusion variation may be matched, according to a regular expression ‘\w+-\w+ fusion’, ‘t\(v;[x\d]+[pq]\d+\.?\d*\)’, ‘rearrang\w+’, or ‘alk\+’, which may resolve to, for example, EGFR-RAD51 fusion, t(v;11q23.3), rearrangement, rearranged, or ALK+.

Once genes and variants have been matched in the text, genes may be further linked to variants at stage 330. Associating Gene to Variant algorithm may connect variants and genes using a word distance algorithm such that for each variant found which is not associated with a gene, the genes which are within a proximity of the variant in the text are matched and checked against the variant. For example, a loop may be inserted to incrementally look each word further from the variant until a match is found. Similarly, drug, effect, and evidence type may be classified according to drug names and drug classes stored in a database or white list and these lists are used to search abstracts for key terms. New drugs may also be annotated as the therapeutic engine searches for a list of pharmaceutical prefixes. In addition, drug “effect” (response, resistance) is also annotated if a drug is found in the abstract or body of the article or publication. Drugs found in the ext may be matched with drug effects, such as Response 371, including response, well-tolerated, benefit, etc., Resistance 372, including resistance, relapsed, progressed, etc., Increase 373 including increase, enhance, improve, prolong, etc., Decrease 374 including decrease, reduce, shorten, poor, etc., Overcome 375 including overcome, target, etc., Activity 376 including activity, efficacy, etc., and Survival 377 including survival, OS, PFS, disease control, etc. If a drug is identified in the abstract or body, the evidence type may be annotated as “therapeutic”. In one example, prognostic entries (outcomes) may search for a different set of key terms, including overall survival, progression free survival, disease free survival, regression free survival, survival, prognosis, prognostic, etc. Disease evidence type may be classified according to an exact match within a whitelist of the feature module from the abstract or body that are present or matched within a relational database such as the NCI Thesaurus.

Once all terms have been matched, they may be provided to structured annotation 380 to generate a complete annotation of the evidence summarized in an abstract or body of an article or publication. A complete annotation may contain content for each of a series of metadata categories. Similar to above, the metadata categories may be linked based upon proximity to each word within the sentence or using an artificial intelligence engine to identify the most likely associations.

Once each metadata has been compiled into a structured format based upon the most likely associations, the evidence may be stored and provided for review.

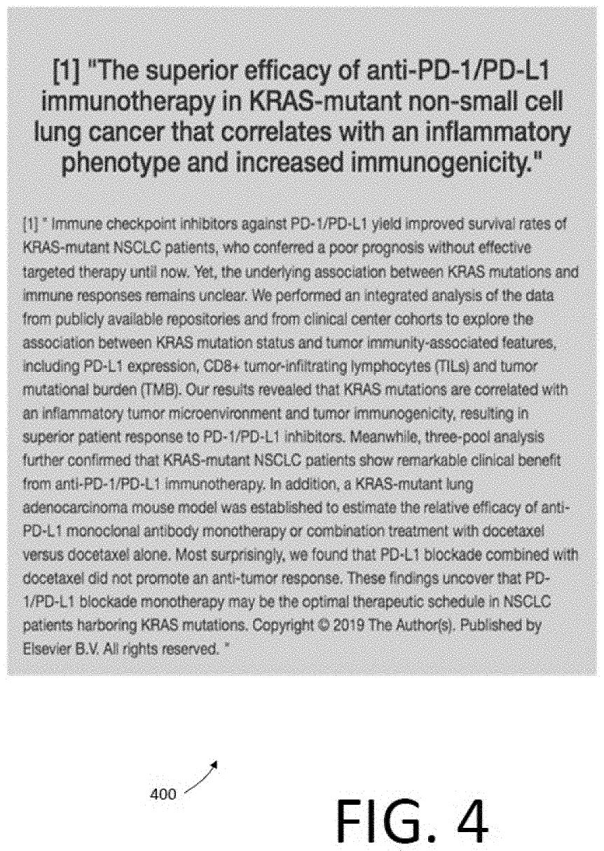

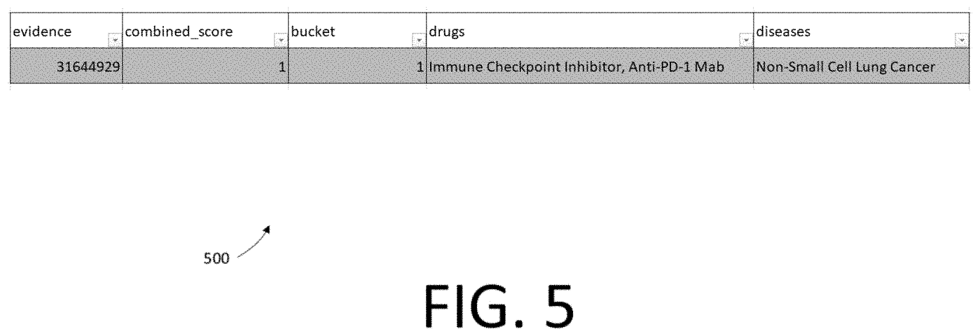

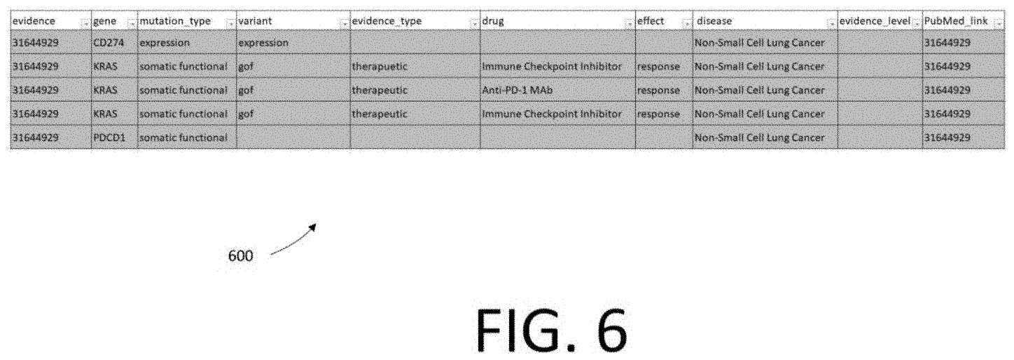

In one example, metadata extraction, such as described with respect to FIGS. 3a and 3b above, may be performed on an abstract of a publication. FIG. 4 illustrates an exemplary article 400, having an abstract and body. The therapy curation engine 100 may analyze the abstract to extract a gene, mutation type, variant, evidence type, disease, drug, and effect from the abstract text. Each extraction may be placed within a metadata category and linked to each other metadata using the complete, structured annotation, as illustrated in FIG. 3b. The one potential combined information that may form a complete annotation for scoring and prioritization is illustrated in FIG. 5. In another example, the therapeutic engine may pre-annotate the article with a plurality of potential annotations such as illustrated in FIG. 6. Lines 1 and 3 in the table have correct annotations that summarize the key result of the article: non-small cell lung cancer tumors with KRAS mutations exhibit response to anti-PD-1/anti-PD-L1 MAb immune checkpoint inhibitors as compared to KRAS wildtype tumors. Since the abstract does not specify exact positional mutations, the therapeutic engine would first look for a positional relationship, and when one did not appear in the text, the engine would annotate the “mutation” category as “KRAS GOF” and “somatic functional” using functional relationships as described with respect to FIG. 3a. In one example, Line 2 may be generated as an incorrect annotation which will be discarded by a curator upon review. Metadata, such as the metadata compiled and annotated in FIGS. 5 and 6, may further include a link to the article or publication or a link to an optical character recognized (OCR) version of the article or publication as text. A viewer, such as software for presenting to a human curator, may enable shorting of columns by selecting a column header and toggling through the sorting direction, including using gestures on a touch pad, or hot keys on a keyboard.

Complete annotations may be sent for ranking or scoring. The therapeutic engine may implement several scoring metrics to determine which articles should be manually reviewed for input into evidence store 150. Each scoring metric may assign an article a score between 0 and 1, where 1 indicates that the article should be included and 0 indicates the article should not be included.

Scoring metrics may include a first scoring method for ranking an article's inclusion through comparing all articles included within the internal database with articles not included in the internal database. In an exemplary embodiment, the first scoring may be referred to as nonhq_score, or non-high quality score, which measures how well the article fits into the internal database based on all internal database articles vs. non-internal database articles. In another exemplary embodiment, a second scoring may include a method for ranking an article's inclusion through comparing only the highest quality of articles of the internal database, the second scoring may be referred to as hq_score, or high quality score, which measures how well the article fits into the internal database based on evidence level >5 internal database articles vs. all other internal database articles. Where an evidence level may identify the quality of the article in relation to the other articles and their level of therapeutic importance to the treatment of a cohort of patients for one or more disease states. In yet another exemplary embodiment, a third scoring may include a method for ranking the accuracy of the metadata extracted from the article, the third scoring may be referred to as a metadata_score. Measuring the quality of the metadata extracted from the article by the metadata extractor may include ranking the articles with more complete annotations higher than articles with missing metadata. In another embodiment, each of the above scoring methods may be combined to generate a weighted average, the combined scoring may be referred to as the combined_score.

Each article identified by source inclusion and exclusion 130 may be scored for suitability for being added to the internal database based on a machine learning classifier to identify a nonhq_score and an hq_score. The inputs of the classifier included the titles and abstracts of a set of articles that are in the internal database and articles that curators have reviewed and determined did not belong in the internal database. In one example, a support vector machines (SVM) may be used for the learning models, for example, to implement a bag-of-words classification mode. The bag-of-words model is a simplifying representation used in natural language processing and information retrieval. In this model, a text is represented as the bag of its words, disregarding grammar and even word order but keeping multiplicity. For example, the bag of words for the article abstract of FIG. 4 may include [the, superior, efficacy, of, anti-PD-1/PD-L1, immunotherapy, in, KRAS-mutant, non-small, cell, lung, cancer, that, correlates, with, an inflammatory, phenotype, and, increased, immunogenicity]. In another example, concepts such as drugs, procedures, therapies, diseases, and effect/outcomes may be treated as a “word.” In one example the bag of concepts may include [the, superior efficacy, of, anti-PD-1/PD-L1 immunotherapy, in, KRAS-mutant, non-small cell lung cancer, that, correlates, with, an inflammatory phenotype, and, increased immunogenicity]. Other variations of the mixed bag of words model may be considered without detracting from the models as described herein. Weights may be assigned to differing terms, words, phrases, or concepts and scores given to a text wherein the score reflects the total weight of the words present in the abstract, including increasing or reducing additional weight of words which repeat more or less frequently. Additional weight may also be assigned to words from articles having a higher evidence score to increase the ranking of articles containing similar words and concepts as presented in the already high scoring articles. Evidence scores may be manually assigned based on their frequency of occurrence in outgoing reports having therapeutic importance. In one example, evidence scores may be assigned by an artificial intelligence engine trained to predict the evidence level based on the frequency of occurrence of the article or publication in outgoing reports to physicians.

For the non_hq and hq prediction scores, training data may reveal a threshold such as an inclusion threshold of 0.5 for which when an hq prediction is greater than 0.5, the expectation should be that there is ˜80% chance the article belongs in the internal database, 20% that it does not; when the hq value is less than 0.5 AND non_hq value is also less than 0.5, the expectation should be that there is a ˜0.5% chance the article belongs in the internal database; and for when the hq value is less than 0.5 AND the non_hq value is greater than 0.5, the expectation should be that there is a ˜50% chance the article belongs in the internal database. Thresholds may be assigned from the classifier or selected by a curator during management of the scoring process.

In one example, articles may be scored on both the title and the abstract. Score predictions for any embodiment may be tuned such that there will be more false positives than false negatives to ensure that potentially therapeutically actionable evidence is not miscategorized or removed from the internal database. A bag of words SVM model as described herein may produce less than 1% false negatives and may be further reduced by combining the scores from multiple methods together.

The metadata_score for an article is computed (e.g., using a computer process) from the annotations identified by the metadata extractor as follows:

Select the annotations with the fewest empty categories

Score each selected annotation from table 1 to generate scores

normalize the scores of selected annotations between 0-1

Each selected annotation is scored by taking a weighted sum of the filled categories and then normalizing the score to be between 0 and 1. The weight for each category is shown in the table below:

TABLE 1

| | | mutation | | | evidence | | | |
|---|---|---|---|---|---|---|---|---|
| | gene | type | mechanism | variant | type | drug | effect | disease |
| weight | 2.04 | 1.44 | 1.44 | 1.44 | 2.41 | 2.50 | 2.52 | 1.03 |

The combined_score for an article is computed as the weighted average of the article's nonhq_score, hq_score, and metadata_score (table 2). The weight for each score is shown in the table below:

TABLE 2

| | nonhq_score | hq_score | metadata_score |
|---|---|---|---|
| weight | 3.45 | 2.70 | 8.66 |

The scores may be bucketed so the most relevant abstracts with a combined score of 0.86-1 appear in bucket 1 and indicate the most likely relevant evidence.

In this example, the therapeutic engine analyzed the abstract in FIG. 5 and scored it with 1, the highest possible score (FIG. 5). This prioritizes the article for the user to curate first as it indicates this article contains highly relevant information.

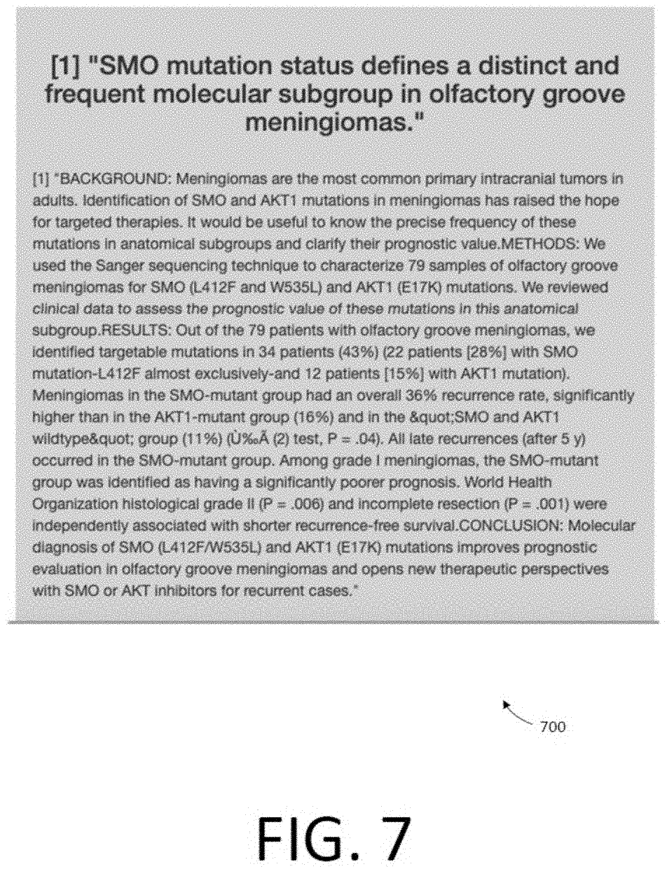

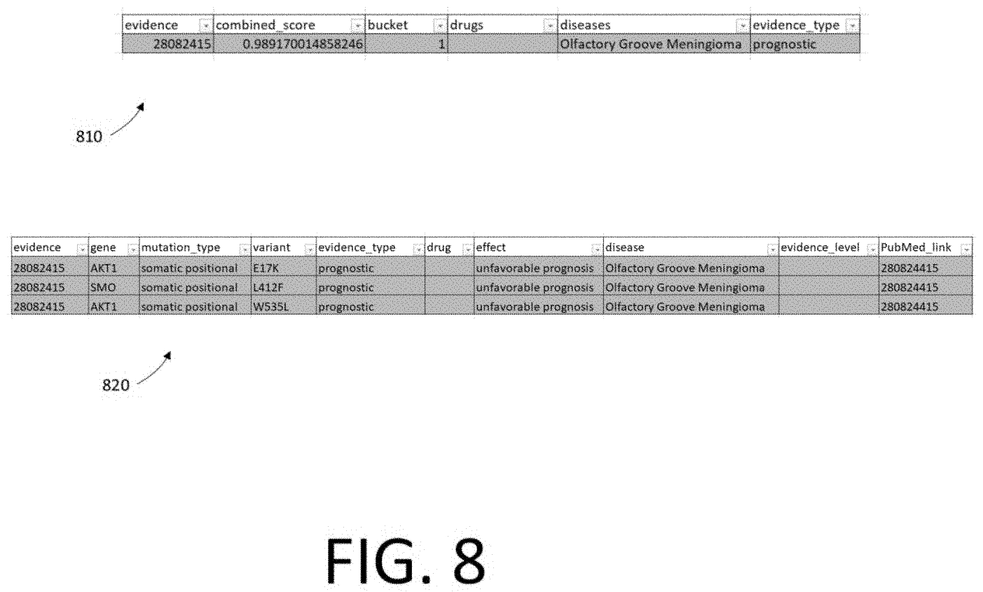

In one example, metadata extraction, such as described with respect to FIGS. 3a and 3b above, may be performed on an abstract of a publication. FIG. 7 illustrates an exemplary article 700, having an abstract and body. The therapy curation engine 100 may analyze the abstract to extract somatic positional variants and prognostic information from the abstract text. Each extraction may be placed within a metadata category and assigned to each other metadata using the complete, structured annotation, as illustrated in FIG. 3b. The one potential combined information that may form a complete annotation for scoring and prioritization is illustrated in FIG. 8, element 810. In another example, the therapeutic engine may pre-annotate the article with a plurality of potential annotations such as illustrated in FIG. 8, element 820.

In FIG. 8, the therapeutic engine predicted 3 annotations for this article with specific positional variants (AKT1 E17K, SMO L412F, and AKT1 W535L). The first 2 gene-variant combinations, AKT1 E17K and SMO L412F, are correctly identified while the third variant (W535L) was incorrectly assigned to AKT1 rather than SMO. A curator may identify the error, correct it, and submit the abstract to retraining with the correction to bolster the artificial intelligence engine performance in the future. In addition, the therapeutic engine annotated “unfavorable prognosis” as the effect for all 3 annotations, but it is only true for SMO variants as per the abstract. Therefore the curator may correct the prognosis and further submit the abstract with the corrections to retraining for the model.

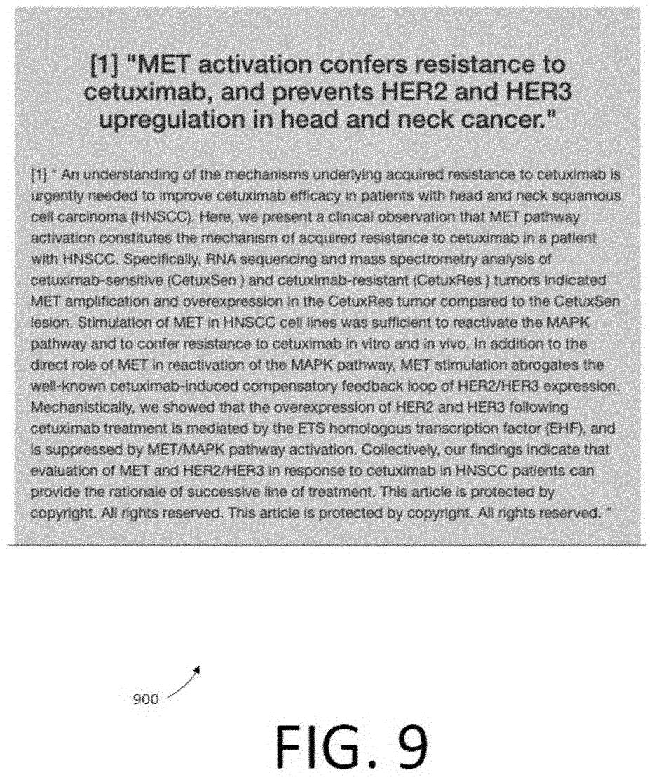

FIG. 9 illustrates an abstract that has both expression and copy number gain evidence for resistance to Cetuximab that may be curated.

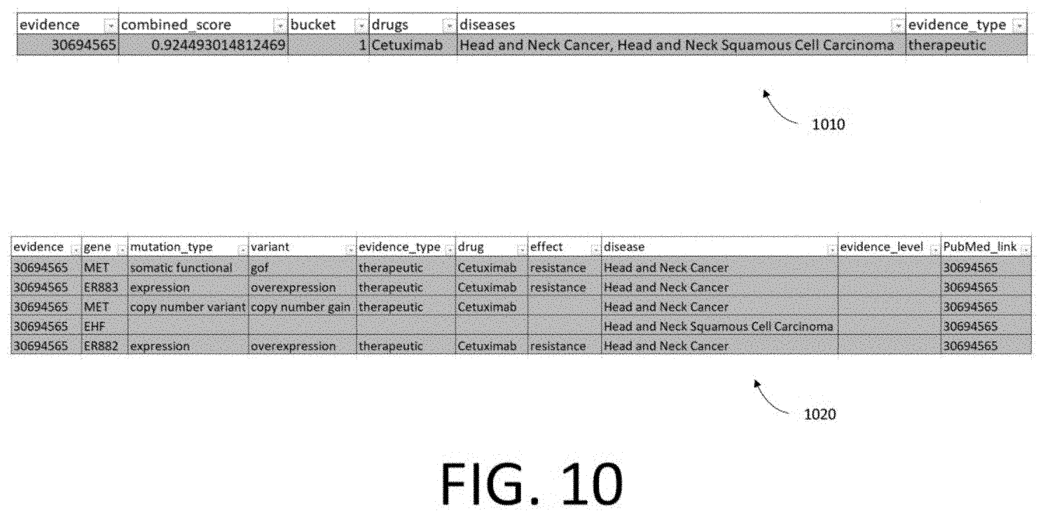

FIG. 10 displays a first predicted annotation at 1010 and 5 predicted annotations for the current example at 1020. The therapeutic engine correctly pulled out the genes, variants, evidence type, drug, disease, and effect from the abstract. However, in this example, none of the predicted annotations are completely correct, where a curator may perform a manual review to complete the annotation process and correct the inline metadata errors.

The therapeutic engine enables prioritization and highlighting of relevant articles, but it may not evaluate the evidence for quality. Therefore, manual review may be performed to read through the articles to identify high quality evidence that is relevant to patients. To facilitate the consistent evaluation of literature, the curator may be presented with a series of questions for each evidence level (clinical research, case study, and preclinical evidence) and type (therapeutic, prognostic). Some examples of points used for quality evaluation include: clinical research: number of patients, criteria used to define response, statistical significance; preclinical research: type of cell line used, assay used to measure drug response, experimental controls; and prognostic evidence: number of patients, criteria used to measure outcome, statistical significance.

In addition, evidence may be given a rating of “good”, “fair”, or “poor” to distinguish its quality among similar studies. This is utilized by the reporting process to select the best pieces of evidence for return on patient reports. In some embodiments, a number of evidence are identified, ranked, and the top N evidence are returned, where N is a threshold number of evidence desired in the reporting. In some examples, this may be 3, in other examples it may be 6, in another it may be uncapped. Some embodiments may include a threshold for the scoring of the evidence that pertains to the reporting, for example, reporting may select all evidence linked to a patient having a score exceeding 0.8, 0.9, 1.0, or any threshold selected from 0-1, based on how many articles are to be linked in the reporting and which evidence should be included.

Therapy Prioritization Engine

In one aspect, the therapy prioritization engine, or therapeutic curation and prioritization module 140 may be a component of a decision assistance machine, specifically an antineoplastic decision assistance machine, and may comprise a variant and disease-aware clinical decision support tool for physicians, such as oncologists. It includes a sophisticated hierarchical disease matching algorithm, variant-specific logic to identify potential therapies, and an explicit rules engine to deliver the best possible potential therapeutic matches per patient.

As input, the therapy prioritization engine receives:

1) The internal database (as explained above and contributed to by the therapeutic engine)
2) The full set of classified variants/alterations in a patient's tumor sequencing
3) The patient disease type

In one embodiment, for each patient variant, the therapy prioritization engine queries the internal database to return variant-specific therapies for the patient. For example, a patient's tumor may contain a mutation at amino-acid position 600 in the BRAF gene that results in the substitution of the amino acid valine (V) with glutamic acid (E), resulting in a ‘V600E’ mutation and specific, directed therapies that are associated with this exact substitution. These specific variant entries are directed specifically at the V600E mutation and are unique from entries that may refer to other, independent mutation in the BRAF gene, for example V600K. The variant matcher thus assures that evidence from the internal database returned to a patient is relevant to their particular tumor. In other embodiments, the therapy prioritization engine may match a patient's variants based on large-scale “gene” matching.

Hierarchical Combination of Gene/Variants

In one embodiment, the system may recognize that newly-received publications may include data that is relevant to the data already extracted from one or more existing articles within the database of publications. For example, a newly-received publication may provide contradictory information relative to the information in an existing publication, and the system may determine whether the newly-received publication should supplant the existing publication(s) completely. Alternatively, the system may evaluate both publications and determine that the newly-received publication is additive with respect to the existing publication(s), for example, by describing a second treatment that can be used in concert with a known treatment identified in an existing publication. Still further, the system may evaluate both publications, determine that one should be considered more authoritative than the other but that both should be presented to a user because the other publication still may have relevance, and then effectuate that presentation in a way that conveys to the user which publication is deemed more authoritative or relevant. The decision support tools within the therapy prioritization engine can include one or more heuristics for evaluating a publication relative to other publications already stored in the database(s) of publication in order to carry out these analyses.

Replacement Heuristic

The therapy prioritization engine may be programmed to evaluate a newly-received publication and determine that it—or by extension, the therapy that it discloses—supplants existing therapy recommendations. In one aspect, the therapy replacement may occur because the new publication identifies deficiencies in an existing therapy as compared to the therapy identified in the publication, identifies a new therapy that provides better results for a class of patients than an existing therapy, identifies a variant-specific therapy for a known mutation that varies from one or more other therapies generally administered in response to the known mutation, etc.

In one embodiment, the replacement heuristic may recognize from a new publication that a therapy directed to a patient with a given variant is ineffective or obsolete when the patient's genome includes a second variant. For example, the system may be encoded to report, based on typical NCCN level evidence, that a patient with a KRAS altered solid tumor cancer should be treated with an EGFR inhibitor, such as Afatinib or Gefitinib. However, the system may ingest a new publication indicating that EGFR inhibitors are less effective or that other therapeutic options are more effective if the patient has KRAS gain of function in combination with MAP3K7 overexpression. For example, that publication may indicate that the overexpression activates an additional WNT pathway that provides a better therapeutic option or that allows for focused targeting of the gene, whereas targeting may be limited to just a pathway in the absence of the MAP3K7 overexpression. In this case, the system may recognize that the original therapy is still valid; it just creates an exception to replace the original therapy with the new/updated therapy when providing recommendations for a patient with the indicated additional variant.

This replacement heuristic may be limited to the specific variant identified in the newly-received publication. Alternatively, the replacement heuristic may be expanded to provide the alternative therapy for users having a class of mutations that have sufficient commonality with the identified mutation. For example, the system may identify a class of variants that behave in a similar fashion to the MAP3K7 overexpression and apply the exception to any patient having a variant within that class. In yet another alternative, the system may bin multiple genes (and, by extension, their variants) into pathways and then indicate that the original and/or updated therapy may be applicable to all genes within that pathway. Thus, in the example above, the system may identify one or more pathways that include KRAS, and then recommend EGFR inhibitors as a therapy for other genes in that one or more pathways, instead of (or in addition to) whatever therapy was previously recommended for variants of those other genes.

Additionally or alternatively, the replacement heuristic may recognize that the presence of a second marker may signify a resistance to the previously-identified primary therapy, i.e., that a therapy identified for a first variant may be rendered obsolete when in the presence of a second variant. For example, the therapy prioritization engine may be programmed to indicate that typical NCCN evidence suggests that lung cancer patients with a range of EGFR activating mutations can be treated with EGFR tyrosine kinase inhibitors (“TKI”s). The system, then, may ingest a publication that indicates that some tumors develop resistance to first-generation EGFR TKIs when the patient also presents with an EGFR T790M point mutation. Thus, when the system is presented with a patient having both EGFR GOF point mutations and an EGFR T790M mutation, the therapy prioritization engine may be programmed to report other TKIs that seem to overcome the T790M resistance and, notably, to not present the first-generation TKIs as recommended therapies. The therapy prioritization engine also may be programmed to affirmatively report the resistance to first-generation TKIs due to the T790M mutation, which may be useful to explain why those standard therapies are not recommended for that particular patient.

The identified resistance may apply to all or part of a therapy for the patient. In the example above, the entire therapy may consist or consist essentially of administering an EGFR TKI. Alternatively, in another example, the therapy may comprise administering an EGFR TKI in combination with a different compound or class of compounds, and the administration of that additional compound(s) may be unaffected by the presence of the additional mutation.

In still another embodiment, the system may recognize several options as viable therapy alternatives. For example, the use of a first-generation EGFR TKI may just be one of several therapies approved for EGFR GOF point mutations, where the T790M mutation may not affect the efficacy of one or more of the other viable therapy alternatives. In that case, the system may replace the first-generation EGFR TKIs as viable therapies with the use of other TKIs and present that alternative alongside the unaffected therapies.

Additive Heuristic

The therapy prioritization engine may be programmed to recognize from a newly-received publication that a plurality of therapies may be used together in response to identification of a particular mutation. In this instance, for example, typical NCCN evidence may suggest that a first therapy be provided in response to identification of a particular mutation. The system then may receive and analyze a new publication from what it determines to be a sufficiently trustable source that indicates a better response (e.g., longer progression free survival rates, lower incidence of side effects, etc.) and may update its programming to report the combination of the first and second therapies when presented with a patient possessing the particular mutation.

In another embodiment, the system may determine from a newly-received publication that a combination of mutations may result in a different suggested therapy, or a particular one out of a plurality of known possible therapies, with a better result than would be the case if the patient presented with only one of the mutations. For example, the system may be programmed to present the combination of APR-246 and azacitidine as the preferred therapy for a TP53 mutation. However, the newly-received publication may include an indication that either a STK11 or EGFR wild type mutation, when present alongside a TP53 mutation, may respond better to anti-PD-1 therapies in lung adenocarcinoma. Thus, the therapy prioritization engine may be configured to present the anti-PD-1 therapy when such a combination of mutations is present. The system also may present the APR-246/azacitidine combination as a possible, albeit less preferred, therapy. In another embodiment, however, the original therapy no longer may be presented as an option, e.g., when the therapeutic benefit of the new therapy is determined to be quantifiably better by some threshold amount than the original therapy, when the new therapy is outlined in a publication deemed more authoritative than the publication reporting the new therapy, and/or when the new therapy is reported in a publication that has an authoritativeness level above some predetermined or user-defined threshold.

In another embodiment, the presence of a first mutation, alone, may correspond to a therapy regimen that comprises administering a first plurality of therapies. Similarly, the presence of a second mutation, alone, may correspond to a therapy regimen that comprises administering a second, different plurality of therapies. When both mutations are present, a publication ingested by the system may indicate that a preferred or most efficacious therapy comprises one or more of the first plurality of therapies with one or more of the second plurality of therapies. In particular, that combination may comprise less than all of at least one of the first and second pluralities of therapies, so that the combination is more than merely combining the two therapies at large.

It should be understood that a “better” result may signify one that is more pertinent or relevant to the patient and not necessarily one that results in an improved outcome or outlook for the patient. In particular, the combination of mutations may cause other information to be conveyed that is different than what would be conveyed if only one of the mutations were present. For example, the therapy prioritization engine may be programmed to indicate a first preferred therapy in the case of KEAP1 loss-of-function and a second preferred therapy in the case of KRAS mutation. When both mutations are present, however, the therapy prioritization engine may draw from a publication that suggests that a co-occurrence of the mutation is an independent factor that predicts shorter survival and a worse prognosis than either mutation alone. In that case, when presented with a patient having both mutations, the system still may present both the first and second therapies as options, but it also may present the reduced outlook information to the user. Preferably, the therapy prioritization engine may present that information before, higher up than, or more conspicuously than the information relating to the first and second therapies.

Prioritization Heuristic

In one embodiment, a patient may present with more than one variant, each of which is associated with its own, separate, independent therapy. The system then may ingest a publication indicating that one of the therapies is more efficacious, has fewer side effects, etc., than the other therapy. Alternatively, each of the therapies may have generally similar efficacies, side effect levels, etc., but one of the publications outlining one of the therapies and its related information may be determined to be more authoritative or otherwise of higher quality evidence. In such situations, the therapy prioritization module may select the “better” therapy in the former case or the therapy from the more authoritative source in the latter case for presentation when the combination of variants is present. In one embodiment, the therapy prioritization module may present the additional therapy in a location or manner that conveys to the user its lower prioritization. In another embodiment, the therapy prioritization module may just not present the additional therapy to the user. In either case, the newly-acquired information may provide a link between the preferred therapy and one or more variants. Alternatively, the publication may indicate a link between the preferred therapy and one or more other patient-identifiable features such as tumor status or staging.

For example, certain tumors affect DNA repair machinery such as homologous recombination or DNA repair pathways. Depending on what mutations are causing those tumors, the patients may be eligible for several different NCCN- or FDA-approved therapies. The system then may ingest a publication that indicates that tumor status generally, or homologous recombination deficiency (HRD+), specifically, may be a more accurate or effective indicator of which therapy to select. Then, when presented with a patient with homologous recombination deficiency, the therapy prioritization model may be programmed to pick a specific one of the possible approved therapies, such as administration of a PARP inhibitor, to present as the preferred therapy for the patient over and/or instead of one or more of the other possible therapies that may be possible due to the patient's identified mutations.

In another example, the therapy prioritization module may ingest a publication with preclinical published evidence suggesting that patients with FGFR2 extracellular domain mutations may benefit from treatment with FGFR inhibitors including infigratinib and ponatinib. At the same time, the therapy prioritization module may be programmed to report that patients with an EGFR activating mutation in lung cancer may benefit from treatments in alignment with NCCN guidelines. The system may ingest a publication indicating that the EGFR-related therapy is more effective, or the system may determine that the publication(s) reporting the EGFR-related therapies are more authoritative than those reporting the use of FGFR inhibitors for FGFR2 mutations and, as a result, may present the NCCN-related therapies in the situation of a patient presenting with both mutations. The system may omit reporting of the FGFR inhibitor-related therapies or, alternatively, may present those therapies but in a manner that conveys their lower prioritization or authoritativeness of their source. In this example, and in general for therapies that are omitted, the system may include an omitted therapies section to which the user may navigate, the omitted therapies section including links to the publications detailing the omitted therapies.

It should be appreciated that there may be overlap among these heuristics and that they may operate together within the therapy prioritization module. For example, a later-received publication that indicates a specific therapy in view of a combination of variants that is different than the suggested therapy for each of those variants may be viewed as triggering the therapy prioritization module to execute the replacement heuristic in that the combination-specific therapy that will be reported may be seen as replacing reporting each of the different, variant-specific therapies. Alternatively, that same process may be characterized as execution of the additive heuristic, since it is the combination of variants that triggers the combination-specific therapy as preferred over the variant-specific ones.

Additionally, although the combinations in the examples discussed above relate to pairings of different mutations, it should be appreciated that the heuristics are not so limited but instead may apply to any combination of the features discussed herein, such as those stored within features modules 110. For example, rather than variant information, the system may rely on biomarker information or demographic information, in combination with information relating to a single variant, to alter the therapy-related information that would be presented without the benefit of that additional biomarker information, demographic information, etc.

The therapy prioritization engine 140, variant matcher, or data-criteria matcher 120 (and system 300 of FIGS. 3a and 3b), may also return actionable implications of interacting variants, i.e. cases where the combination of two or more variants in a patient has an implication that differs from any single variant by itself. In these cases of variant-variant interactions, single gene or even specific variant matching does not adequately provide the best possible precision therapeutics for a patient. For example, by itself a loss-of-function mutation in the KEAP1 gene in a lung cancer does not suggest treatment with any drugs, but if the same patient's tumor also contains a gain-of-function mutation in the KRAS gene, there are therapies and prognostic associations associated with the interaction of the two variants that are not relevant for either variant independently. Many examples of these variant-variant interactions and therapeutic implications are present and curated in the internal database. These interacting associations are curated and stored in the internal database and the therapy prioritization engine 140, and system of FIGS. 3a and 3b, variant matcher will provide these associations given only the case where both variants are present in a patient's tumor and prioritize such interactions over conflicting non-interacting evidence.

These variant interactions and the actionable evidence associated with them become even more important when examined in the context of acquired drug resistance. In one canonical example, patients with actionable mutations in the EGFR gene can be treated with first-generation Tyrosine Kinase Inhibitors (TKIs) as a standard-of-care. But in response to treatment, these tumors often develop a secondary acquired resistance mutation in EGFR that renders this first line of TKIs ineffective. In this case, the patient will have two actionable alterations in EGFR. The first that is known to respond to one mode of treatment, and the second that is known to be resistant to the first mode of treatment but may respond to other regimens. Taken independently, these two EGFR alterations suggest entirely different and sometimes conflicting treatment options. But analyzed in the context of a variant-variant interaction, it becomes clear that therapeutics and prognoses from the second, acquired-resistance, alteration should be prioritized over the first.

After the variant matcher returns all specific actionable entries from the internal database, the therapy prioritization engine may score those entries based on the similarity of the evidence to the patient disease and the strength of the evidence supporting the assertion. In this aspect, rather than simply returning all evidence unweighted by how closely the evidence matches a patient's disease, the therapy prioritization engine may make use of hierarchical clustering of diseases to score how similar a patient's disease is to a piece of evidence in the internal database. This disease matcher, such as data-criteria matching module 120, may make use of a hierarchical system of disease encoding to match a patient disease to internal database disease based on how closely related the two diseases are. The therapy matcher assigns each variant-matched entry a therapy score from 0-1 based on how well the patient diseases matches the internal database entry disease. Additional scores from 0-1 are assigned for (1) the evidence-level of the internal database entry assertion and (2) the FDA approval status of the drug in question. These three factors, and potentially others, then combine to form a single therapy score for the entry in question given the patient disease.

Finally, given all of these scores for every patient variant, the therapy prioritization engine 140, and system of FIGS. 3a and 3b, may apply a set of manually curated rules to determine which entries should be returned for a particular patient. This step ensures that we have a consistent, robust, and clinically rational reason for including particular pieces of evidence on a patient report. For some processes, running a black-box machine learning algorithm may shroud the reasons behind an inclusion or exclusion of an article in mystery; however, with hard rules, the rationale why particular evidence is included or excluded per patient is readily understood from the applied ruleset.

FIG. 3b displays a representation of an exemplary therapy prioritization engine. Variant matcher, such as variant and mutation disambiguation 320 and Match variant and gene 330, may match a patient variant to one or more variants from internal database, or gene store 110. The variant matcher may allow for gene equivalence matching. For example, the variant matcher may allow for matching genes having a symbol, to a geneID, intresID, or to a specific chromosomal and loci position pairing to a gene at the same location. The variant matcher may also allow for specificity beyond gene equivalence matching. In one example, the variant matcher permits the automatic identification of interacting variants by referencing one or more interacting variants from a whitelist.

Disease matcher may be utilized to indicate how well an entry in an internal database matches a patient's disease. For instance, the disease matcher may leverage a disease ontology, such as the NCI Thesaurus (available at http://obofoundry.org/ontology/ncit.html and incorporated herein by reference) disease ontology, to score how well an entry from the internal database matches to a patient disease. The disease matcher also allows for more specific therapeutic recommendations. As detailed herein for the ranking (score), similarity between patient's disease and disease in the entry is utilized to return the most specific entry. Cohorts of similar disease types not captured in the NCI thesaurus were also added to the logic to include additional disease state that appear in a patient database. For example, cancer types that are impacted by hormonal signaling pathways, such as breast, prostate, and endometrial cancers, may be cohorted together as “Hormone Sensitive Cancers.” Thus if there are no entries in internal database for the patient's exact disease type, disease matcher is able to prioritize an entry of a more-similar cancer type. In one aspect, therapies that are recommended, such as through clinical practice guidelines like NCCN guidelines, may be matched with RNA expression data to further elucidate these similar cancer groupings, as defined by diseases with similar RNA expression profiles that are treated similarly in the field.

Reporting ruleset, such as rule-based selection of therapeutic curation and prioritization may include a set of rules identifying the circumstances under which therapies are excluded from the report. The ruleset may include five categories of exclusion rules, including: disease distinction: rules that ensure therapies specific to certain disease types are not returned inappropriately; resistance/non-response: rules specifying situations where resistance and non-response to therapies should or should not be returned; prognostic: rules dictating when prognostic evidence is appropriate; drug redundancy: rules to ensure the same drug or drugs of the same class are not over-returned; and best evidence: rules governing how the tool should determine what the highest quality evidence is.

Therapy prioritization engine may be integrated into a report generation pipeline. For instance, each patient's SNV/indel, CNV, RNA, and fusion classifications may be run through the therapy prioritization engine to determine the best therapy recommendations for the patient. Rather than relying on static templates, the therapy prioritization engine may allow for variable and distinct recommendations based on the entire genetic profile of the tumor and the exact disease type.

Internal Reference Database/Knowledge Database

The knowledge database may comprise abstracted information about medical and/or scientific publications. The internal database may characterize publications by various dimensions and/or labels, such as the level of evidence (e.g. whether the publication is from clinical practice guidelines; from evidence used to support a regulatory decision, such as a FDA decision; from clinical research; from case studies; or from pre-clinical research). The internal database may characterize publications by whether they are appropriate for clinical consideration or for scientific consideration. For instance, the internal database may characterize a publication as appropriate for clinical consideration if it is from clinical practice guidelines such as, in the case of oncology, NCCN guidelines; from FDA evidence; or from clinical research. The internal database may characterize a publication for scientific consideration if it reflects experimental research, such as pre-clinical research; preliminary prognosis evidence; conflicting evidence; or case studies.

The internal database may employ the use of one or more evidence and reporting templates, where reporting templates may supply a combination of words or words and graphics to a report that indicate the suitability of a therapy for the respective patient. A template may include a pre-created set of therapeutic, prognostic, and/or diagnostic evidence that is matched to a listing of data elements, such as genotypic, phenotypic, and/or other clinical or molecular information relevant to a particular patient's care. For example, a template for oncology publications may include pre-created sets of therapeutic, prognostic, and/or diagnostic evidence that is matched to a specific gene, cancer type, and variant. A template may be more specific or more general, depending on the circumstance of its use in any particular application. For example, a more specific template may include a specific gene, specific mutation, and specific cancer subtype (e.g. a template for EGFR T790M in non small-cell lung cancer). A more general subtype may include less specificity with respect to one or more data elements. For example, a more general template may include a specific gene but be less specific in other data elements (e.g. a template for PTEN loss-of-function in solid tumors).

Templates in Table 3 identify a number of different solid tumors or tumor tissue types.

TABLE 3

| Template Name | Gene | Variant type | Diseases included |
|---|---|---|---|
| 10.2_CDKN2A_general_CNL | CDKN2A | Copy number loss | Ovarian Cancer, Cervical Cancer, |
| | | | Colorectal Cancer, Endocrine |
| | | | Tumor, Oropharyngeal Cancer, |
| | | | Retinoblastoma, Adrenal cancer, |
| | | | Neural, Basal Cell Carcinoma, |
| | | | Breast Cancer, Non-Clear Cell |
| | | | Renal Cell Carcinoma, Tumor of |
| | | | Unknown Origin, Gastrointestinal |
| | | | Stromal Tumor, Bladder Cancer, |
| | | | Gastric Cancer, Bone Cancer, Non- |
| | | | Small Cell Lung Cancer, |
| | | | Thymoma, Prostate Cancer, Skin |
| | | | Cancer, Thyroid Cancer, Sarcoma, |
| | | | Testicular cancer, Head and Neck |
| | | | Cancer, Head and Neck Squamous |
| | | | Cell Carcinoma, Meningioma, |
| | | | Peritoneal cancer, Endometrial |
| | | | Cancer, Mesothelioma, Esophageal |
| | | | Cancer, Small Cell Lung Cancer |
| 100_HER2_GOF_general | ERBB2 | Gain-of-function | Ovarian Cancer, Cervical Cancer, |
| | (HER2) | | Colorectal Cancer, Endocrine |
| | | | Tumor, Biliary Cancer, Melanoma, |
| | | | Tumor of Unknown Origin, Kidney |
| | | | Cancer, Bladder Cancer, Gastric |
| | | | Cancer, Prostate Cancer, Skin |
| | | | Cancer, Sarcoma, Head and Neck |
| | | | Cancer, Head and Neck Squamous |
| | | | Cell Carcinoma, Endometrial |
| | | | Cancer, Pancreatic Cancer, |
| | | | Esophageal Cancer, Small Cell |
| | | | Lung Cancer |
| 311_TSC1_general_LOF | TSC1 | Loss-of-function | Ovarian Cancer, Cervical Cancer, |
| | | | Uveal Melanoma, Colorectal |
| | | | Cancer, Liver Cancer, Endocrine |
| | | | Tumor, Oropharyngeal Cancer, |
| | | | Retinoblastoma, Biliary Cancer, |
| | | | Basal Cell Carcinoma, Breast |
| | | | Cancer, Melanoma, Glioblastoma, |
| | | | Tumor of Unknown Origin, |
| | | | Gastrointestinal Stromal Tumor, |
| | | | Medulloblastoma, Bladder Cancer, |
| | | | Gastric Cancer, Bone Cancer, Non- |
| | | | Small Cell Lung Cancer, |
| | | | Thymoma, Low Grade Glioma, |
| | | | Prostate Cancer, Skin Cancer, |
| | | | Thyroid Cancer, Sarcoma, |
| | | | Testicular cancer, Head and Neck |
| | | | Cancer, Head and Neck Squamous |
| | | | Cell Carcinoma, Meningioma, |
| | | | Peritoneal cancer, Endometrial |
| | | | Cancer, Pancreatic Cancer, |
| | | | Mesothelioma, Esophageal Cancer, |
| | | | Small Cell Lung Cancer |
| 108.2_IDH1_GOF_general | IDH1 | Gain-of-function | Ovarian Cancer, Cervical Cancer, |
| (not Brain) | | at codon 132 | Colorectal Cancer, Chromophobe |
| | | | Renal Cell Carcinoma, Liver |
| | | | Cancer, Endocrine Tumor, |
| | | | Oropharyngeal Cancer, |
| | | | Retinoblastoma, Adrenal cancer, |
| | | | Breast Cancer, Melanoma, Non- |
| | | | Clear Cell Renal Cell Carcinoma, |
| | | | Tumor of Unknown Origin, Kidney |
| | | | Cancer, Bladder Cancer, Gastric |
| | | | Cancer, Bone Cancer, Non-Small |
| | | | Cell Lung Cancer, Thymoma, |
| | | | Prostate Cancer, Clear Cell Renal |
| | | | Cell Carcinoma, Skin Cancer, |
| | | | Thyroid Cancer, Sarcoma, |
| | | | Testicular cancer, Head and Neck |
| | | | Cancer, Head and Neck Squamous |
| | | | Cell Carcinoma, Meningioma, |
| | | | Peritoneal cancer, Pancreatic |
| | | | Cancer, Esophageal Cancer |
| 113_RB1_general | RB1 | Copy number loss | Ovarian Cancer, Cervical Cancer, |
| (CNL) | | | Colorectal Cancer, Chromophobe |
| | | | Renal Cell Carcinoma, Liver |
| | | | Cancer, Endocrine Tumor, |
| | | | Oropharyngeal Cancer, |
| | | | Retinoblastoma, Biliary Cancer, |
| | | | Adrenal cancer, Melanoma, Non- |
| | | | Clear Cell Renal Cell Carcinoma, |
| | | | Tumor of Unknown Origin, Kidney |
| | | | Cancer, Gastric Cancer, Bone |
| | | | Cancer, Non-Small Cell Lung |
| | | | Cancer, Thymoma, Prostate |
| | | | Cancer, Clear Cell Renal Cell |
| | | | Carcinoma, Skin Cancer, Thyroid |
| | | | Cancer, Sarcoma, Testicular |
| | | | cancer, Head and Neck Cancer, |
| | | | Head and Neck Squamous Cell |
| | | | Carcinoma, Meningioma, |
| | | | Peritoneal cancer, Endometrial |
| | | | Cancer, Pancreatic Cancer, |
| | | | Esophageal Cancer |
| 12_EGFR_CNG_general | EGFR | Copy number gain | Ovarian Cancer, Cervical Cancer, |
| | | | Chromophobe Renal Cell |
| | | | Carcinoma, Liver Cancer, |
| | | | Endocrine Tumor, Oropharyngeal |
| | | | Cancer, Retinoblastoma, Biliary |
| | | | Cancer, Adrenal cancer, Breast |
| | | | Cancer, Melanoma, Non-Clear Cell |
| | | | Renal Cell Carcinoma, Tumor of |
| | | | Unknown Origin, Kidney Cancer, |
| | | | Bladder Cancer, Bone Cancer, |
| | | | Non-Small Cell Lung Cancer, |
| | | | Thymoma, Prostate Cancer, Clear |
| | | | Cell Renal Cell Carcinoma, Skin |
| | | | Cancer, Thyroid Cancer, Sarcoma, |
| | | | Testicular cancer, Head and Neck |
| | | | Cancer, Meningioma, Peritoneal |
| | | | cancer, Endometrial Cancer, |
| | | | Pancreatic Cancer, Small Cell Lung |
| | | | Cancer |
| 131.3_KRAS_GOF_SolidTumorGeneral | KRAS | Gain-of-function | Ovarian Cancer, Cervical Cancer, |
| | | at codons 12, 13, | Chromophobe Renal Cell |
| | | 14, 19, 22, 60, 61, | Carcinoma, Endocrine Tumor, |
| | | 117, or 146 | Oropharyngeal Cancer, |
| | | | Retinoblastoma, Adrenal cancer, |
| | | | Brain Cancer, Melanoma, Non- |
| | | | Clear Cell Renal Cell Carcinoma, |
| | | | Glioblastoma, Tumor of Unknown |
| | | | Origin, Kidney Cancer, Bladder |
| | | | Cancer, Bone Cancer, Thymoma, |
| | | | Low Grade Glioma, Prostate |
| | | | Cancer, Clear Cell Renal Cell |
| | | | Carcinoma, Skin Cancer, Thyroid |
| | | | Cancer, Sarcoma, Testicular |
| | | | cancer, Head and Neck Cancer, |
| | | | Head and Neck Squamous Cell |
| | | | Carcinoma, Meningioma, |
| | | | Peritoneal cancer, Mesothelioma, |
| | | | Esophageal Cancer, Small Cell |
| | | | Lung Cancer |
| 220_ARID1A_general_LOF | ARID1A | Loss-of-function | Cervical Cancer, Colorectal |
| | | | Cancer, Chromophobe Renal Cell |
| | | | Carcinoma, Liver Cancer, |
| | | | Endocrine Tumor, Oropharyngeal |
| | | | Cancer, Retinoblastoma, Biliary |
| | | | Cancer, Adrenal cancer, Basal Cell |
| | | | Carcinoma, Brain Cancer, Breast |
| | | | Cancer, Melanoma, Non-Clear Cell |
| | | | Renal Cell Carcinoma, |
| | | | Glioblastoma, Tumor of Unknown |
| | | | Origin, Kidney Cancer, |
| | | | Gastrointestinal Stromal Tumor, |
| | | | Medulloblastoma, Bladder Cancer, |
| | | | Gastric Cancer, Non-Small Cell |
| | | | Lung Cancer, Low Grade Glioma, |
| | | | Prostate Cancer, Clear Cell Renal |
| | | | Cell Carcinoma, Skin Cancer, |
| | | | Thyroid Cancer, Sarcoma, |
| | | | Testicular cancer, Head and Neck |
| | | | Cancer, Head and Neck Squamous |
| | | | Cell Carcinoma, Meningioma, |
| | | | Endometrial Cancer, Pancreatic |
| | | | Cancer, Mesothelioma, Esophageal |
| | | | Cancer, Small Cell Lung Cancer |
| 161.1_BRCA1_CNL_Solid_Tumor | BRCA1 | Copy number loss | Colorectal Cancer, Endocrine |
| | | | Tumor, Biliary Cancer, Tumor of |
| | | | Unknown Origin, Gastric Cancer, |
| | | | Non-Small Cell Lung Cancer, Head |
| | | | and Neck Cancer, Head and Neck |
| | | | Squamous Cell Carcinoma, |
| | | | Endometrial Cancer, Esophageal |
| | | | Cancer |
| 187_CDK4_CNG_solid_tumor | CDK4 | Copy number gain | Ovarian Cancer, Cervical Cancer, |
| | | | Colorectal Cancer, Chromophobe |
| | | | Renal Cell Carcinoma, Liver |
| | | | Cancer, Endocrine Tumor, |
| | | | Oropharyngeal Cancer, |
| | | | Retinoblastoma, Biliary Cancer, |
| | | | Adrenal cancer, Breast Cancer, |
| | | | Melanoma, Non-Clear Cell Renal |
| | | | Cell Carcinoma, Glioblastoma, |
| | | | Tumor of Unknown Origin, Kidney |
| | | | Cancer, Bladder Cancer, Gastric |
| | | | Cancer, Bone Cancer, Non-Small |
| | | | Cell Lung Cancer, Thymoma, Low |
| | | | Grade Glioma, Prostate Cancer, |
| | | | Clear Cell Renal Cell Carcinoma, |
| | | | Skin Cancer, Thyroid Cancer, |
| | | | Sarcoma, Testicular cancer, Head |
| | | | and Neck Cancer, Head and Neck |
| | | | Squamous Cell Carcinoma, |
| | | | Meningioma, Peritoneal cancer, |
| | | | Endometrial Cancer, Pancreatic |
| | | | Cancer, Mesothelioma, Esophageal |
| | | | Cancer, Small Cell Lung Cancer |
| 91.3_PIK3CA_GOF_Solid_tumor | PIK3CA | Gain-of-function | Chromophobe Renal Cell |
| | | | Carcinoma, Liver Cancer, |
| | | | Endocrine Tumor, Oropharyngeal |
| | | | Cancer, Retinoblastoma, Biliary |
| | | | Cancer, Adrenal cancer, Basal Cell |
| | | | Carcinoma, Melanoma, Non-Clear |
| | | | Cell Renal Cell Carcinoma, Tumor |
| | | | of Unknown Origin, Kidney |
| | | | Cancer, Gastrointestinal Stromal |
| | | | Tumor, Bladder Cancer, Gastric |
| | | | Cancer, Bone Cancer, Thymoma, |
| | | | Prostate Cancer, Clear Cell Renal |
| | | | Cell Carcinoma, Skin Cancer, |
| | | | Thyroid Cancer, Sarcoma, |
| | | | Testicular cancer, Head and Neck |
| | | | Cancer, Head and Neck Squamous |
| | | | Cell Carcinoma, Peritoneal cancer, |
| | | | Pancreatic Cancer, Mesothelioma, |
| | | | Esophageal Cancer, Small Cell |
| | | | Lung Cancer |
| 55_EML4 (or other)- | ALK | ALK fusion | Biliary Cancer, Bladder Cancer, |
| ALK_Fusion | | | Breast Cancer, Cervical Cancer, |
| | | | Chromophobe Renal Cell |
| | | | Carcinoma, Clear Cell Renal Cell |
| | | | Carcinoma, Endometrial Cancer, |
| | | | Esophageal Cancer, Gastric Cancer, |
| | | | Head and Neck Cancer, Head and |
| | | | Neck Squamous Cell Carcinoma, |
| | | | Liver Cancer, Low Grade Glioma, |
| | | | Melanoma, Meningioma, Non- |
| | | | Clear Cell Renal Cell Carcinoma, |
| | | | Non-Small Cell Lung Cancer, |
| | | | Oropharyngeal Cancer, Ovarian |
| | | | Cancer, Pancreatic Cancer, |
| | | | Retinoblastoma, Sarcoma, |
| | | | Testicular cancer, Thyroid Cancer, |
| | | | Kidney Cancer, Skin Cancer |
| 630_any5′_3′NRG1_solid_tumor | NRG1 | NRG1 fusion | Biliary Cancer, Bladder Cancer, |
| | | | Breast Cancer, Cervical Cancer, |
| | | | Chromophobe Renal Cell |
| | | | Carcinoma, Clear Cell Renal Cell |
| | | | Carcinoma, Colorectal Cancer, |
| | | | Endometrial Cancer, Esophageal |
| | | | Cancer, Gastric Cancer, Head and |
| | | | Neck Cancer, Head and Neck |
| | | | Squamous Cell Carcinoma, Liver |
| | | | Cancer, Low Grade Glioma, |
| | | | Melanoma, Meningioma, Non- |
| | | | Clear Cell Renal Cell Carcinoma, |
| | | | Oropharyngeal Cancer, Ovarian |
| | | | Cancer, Pancreatic Cancer, |
| | | | Retinoblastoma, Sarcoma, |
| | | | Testicular cancer, Thyroid Cancer, |
| | | | Kidney Cancer, Skin Cancer |
| 631_any5′_NTRK_fusion_general | NTRK1, | NTRK1/2/3 | Biliary Cancer, Bladder Cancer, |
| | NTRK2, | fusions | Breast Cancer, Cervical Cancer, |
| | NTRK3 | | Chromophobe Renal Cell |
| | | | Carcinoma, Clear Cell Renal Cell |
| | | | Carcinoma, Endometrial Cancer, |
| | | | Esophageal Cancer, Gastric Cancer, |
| | | | Head and Neck Cancer, Head and |
| | | | Neck Squamous Cell Carcinoma, |
| | | | Liver Cancer, Low Grade Glioma, |
| | | | Melanoma, Meningioma, Non- |
| | | | Clear Cell Renal Cell Carcinoma, |
| | | | Oropharyngeal Cancer, Ovarian |
| | | | Cancer, Pancreatic Cancer, |
| | | | Retinoblastoma, Sarcoma, |
| | | | Testicular cancer, Thyroid Cancer, |
| | | | Kidney Cancer, Skin Cancer, |
| | | | Prostate Cancer |
| 212_MAP2K4_LOF_general | MAP2K4 | Loss-of-function | Ovarian Cancer, Cervical Cancer, |
| | | | Colorectal Cancer, Chromophobe |
| | | | Renal Cell Carcinoma, Liver |
| | | | Cancer, Endocrine Tumor, |
| | | | Oropharyngeal Cancer, |
| | | | Retinoblastoma, Biliary Cancer, |
| | | | Adrenal cancer, Breast Cancer, |
| | | | Melanoma, Non-Clear Cell Renal |
| | | | Cell Carcinoma, Glioblastoma, |
| | | | Tumor of Unknown Origin, Kidney |
| | | | Cancer, Bladder Cancer, Gastric |
| | | | Cancer, Bone Cancer, Non-Small |
| | | | Cell Lung Cancer, Thymoma, |
| | | | Prostate Cancer, Clear Cell Renal |
| | | | Cell Carcinoma, Skin Cancer, |
| | | | Thyroid Cancer, Sarcoma, |
| | | | Testicular cancer, Head and Neck |
| | | | Cancer, Head and Neck Squamous |
| | | | Cell Carcinoma, Meningioma, |
| | | | Peritoneal cancer, Endometrial |
| | | | Cancer, Pancreatic Cancer, |
| | | | Esophageal Cancer |
| 218_TP53_R175_solid_tumors | TP53 | Loss-of-function | Cervical Cancer, Uveal Melanoma, |
| | | at codon 175 | Colorectal Cancer, Chromophobe |
| | | | Renal Cell Carcinoma, Liver |
| | | | Cancer, Endocrine Tumor, |
| | | | Oropharyngeal Cancer, |
| | | | Retinoblastoma, Biliary Cancer, |
| | | | Adrenal cancer, Neural, |
| | | | Neuroblastoma, Basal Cell |
| | | | Carcinoma, Brain Cancer, Breast |
| | | | Cancer, Melanoma, Non-Clear Cell |
| | | | Renal Cell Carcinoma, |
| | | | Glioblastoma, Tumor of Unknown |
| | | | Origin, Kidney Cancer, |
| | | | Medulloblastoma, Bladder Cancer, |
| | | | Gastric Cancer, Bone Cancer, Non- |
| | | | Small Cell Lung Cancer, |
| | | | Thymoma, Low Grade Glioma, |
| | | | Prostate Cancer, Clear Cell Renal |
| | | | Cell Carcinoma, Skin Cancer, |
| | | | Thyroid Cancer, Sarcoma, |
| | | | Testicular cancer, Head and Neck |
| | | | Cancer, Head and Neck Squamous |
| | | | Cell Carcinoma, Meningioma, |
| | | | Peritoneal cancer, Endometrial |
| | | | Cancer, Pancreatic Cancer, |
| | | | Mesothelioma, Esophageal Cancer, |
| | | | Small Cell Lung Cancer |
| 264_PTEN_general | PTEN | Loss-of-function | Colorectal Cancer, Chromophobe |
| (LOF) | | | Renal Cell Carcinoma, Liver |
| | | | Cancer, Endocrine Tumor, |
| | | | Oropharyngeal Cancer, |
| | | | Retinoblastoma, Biliary Cancer, |
| | | | Adrenal cancer, Melanoma, Non- |
| | | | Clear Cell Renal Cell Carcinoma, |
| | | | Tumor of Unknown Origin, Kidney |
| | | | Cancer, Gastrointestinal Stromal |
| | | | Tumor, Medulloblastoma, Bladder |
| | | | Cancer, Gastric Cancer, Bone |
| | | | Cancer, Non-Small Cell Lung |
| | | | Cancer, Thymoma, Clear Cell |
| | | | Renal Cell Carcinoma, Skin |
| | | | Cancer, Thyroid Cancer, Sarcoma, |
| | | | Testicular cancer, Head and Neck |
| | | | Cancer, Head and Neck Squamous |
| | | | Cell Carcinoma, Meningioma, |
| | | | Peritoneal cancer, Pancreatic |
| | | | Cancer, Esophageal Cancer, Small |
| | | | Cell Lung Cancer |
| 360_MTOR_GOF_General | MTOR | Gain-of-function | Ovarian Cancer, Cervical Cancer, |
| | | | Colorectal Cancer, Liver Cancer, |
| | | | Endocrine Tumor, Oropharyngeal |
| | | | Cancer, Retinoblastoma, Biliary |
| | | | Cancer, Adrenal cancer, Brain |
| | | | Cancer, Breast Cancer, Melanoma, |
| | | | Glioblastoma, Gastrointestinal |
| | | | Stromal Tumor, Bladder Cancer, |
| | | | Gastric Cancer, Bone Cancer, Non- |
| | | | Small Cell Lung Cancer, |
| | | | Thymoma, Low Grade Glioma, |
| | | | Prostate Cancer, Skin Cancer, |
| | | | Thyroid Cancer, Sarcoma, |
| | | | Testicular cancer, Head and Neck |
| | | | Cancer, Head and Neck Squamous |
| | | | Cell Carcinoma, Meningioma, |
| | | | Peritoneal cancer, Endometrial |
| | | | Cancer, Pancreatic Cancer, |
| | | | Esophageal Cancer |
| 429_KIT_exon11_general | KIT | Gain-of-function | Ovarian Cancer, Cervical Cancer, |
| | | in exon 11 | Uveal Melanoma, Colorectal |
| | | | Cancer, Chromophobe Renal Cell |
| | | | Carcinoma, Liver Cancer, |
| | | | Oropharyngeal Cancer, |
| | | | Retinoblastoma, Biliary Cancer, |
| | | | Basal Cell Carcinoma, Breast |
| | | | Cancer, Non-Clear Cell Renal Cell |
| | | | Carcinoma, Tumor of Unknown |
| | | | Origin, Kidney Cancer, Bladder |
| | | | Cancer, Gastric Cancer, Bone |
| | | | Cancer, Non-Small Cell Lung |
| | | | Cancer, Prostate Cancer, Clear Cell |
| | | | Renal Cell Carcinoma, Thyroid |
| | | | Cancer, Sarcoma, Testicular |
| | | | cancer, Head and Neck Cancer, T |
| | | | Cell Lymphoma, Head and Neck |
| | | | Squamous Cell Carcinoma, |
| | | | Peritoneal cancer, Endometrial |
| | | | Cancer, Pancreatic Cancer, |
| | | | Esophageal Cancer, Small Cell |
| | | | Lung Cancer |
| 439_FLCN_LOF(w/ | FLCN | Loss-of-function | Ovarian Cancer, Cervical Cancer, |
| TSC2 LOF)_general | | with concomitant | Uveal Melanoma, Colorectal |
| solid tumor | | TSC2 loss-of- | Cancer, Chromophobe Renal Cell |
| | | function | Carcinoma, Liver Cancer, |
| | | | Endocrine Tumor, Oropharyngeal |
| | | | Cancer, Retinoblastoma, Biliary |
| | | | Cancer, Adrenal cancer, Neural, |
| | | | Neuroblastoma, Basal Cell |
| | | | Carcinoma, Brain Cancer, Breast |
| | | | Cancer, Melanoma, Non-Clear Cell |
| | | | Renal Cell Carcinoma, |
| | | | Glioblastoma, Kidney Cancer, |
| | | | Gastrointestinal Stromal Tumor, |
| | | | Medulloblastoma, Bladder Cancer, |
| | | | Gastric Cancer, Bone Cancer, Non- |
| | | | Small Cell Lung Cancer, Low |
| | | | Grade Glioma, Prostate Cancer, |
| | | | Clear Cell Renal Cell Carcinoma, |
| | | | Skin Cancer, Thyroid Cancer, |
| | | | Sarcoma, Testicular cancer, Head |
| | | | and Neck Cancer, Head and Neck |
| | | | Squamous Cell Carcinoma, |
| | | | Meningioma, Peritoneal cancer, |
| | | | Endometrial Cancer, Pancreatic |
| | | | Cancer, Mesothelioma, Esophageal |
| | | | Cancer, Small Cell Lung Cancer |

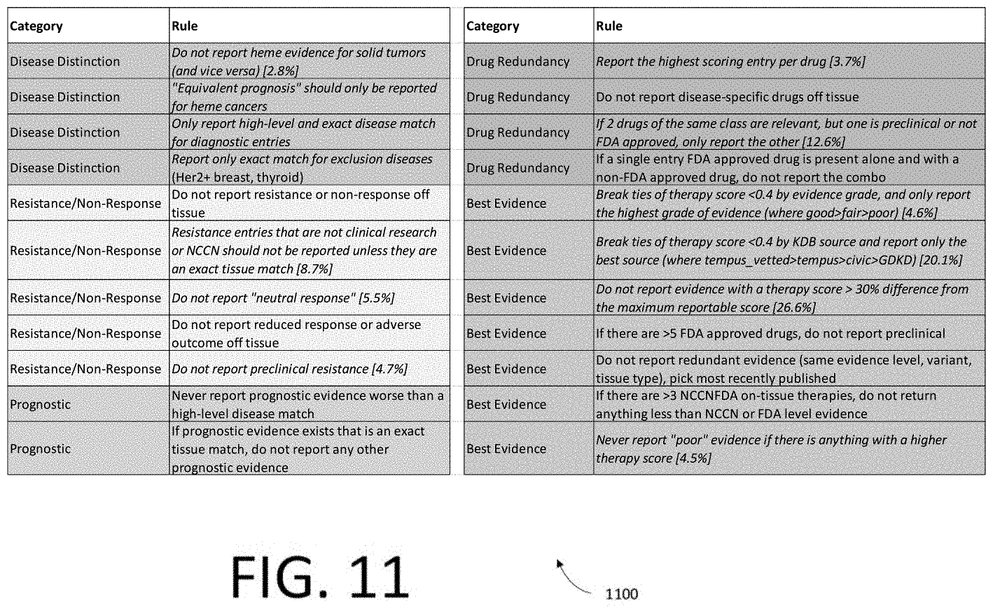

FIG. 11 illustrates a rule-based selection for identifying which evidence should be stored in the internal database, according to an embodiment of the invention.

The results of the variant matcher, disease matcher, and rule-based ruleset may be combined to form an evidence score/ranking without the artificial intelligence engine.

In another aspect, a therapy prioritization engine may operate as a weighted decision model for therapy scoring. For instance, the engine may return a therapy score equal to a weighted sum of a disease score, an evidence level, and a regulatory approval. In one example, the Therapy Score=(0.7*Disease Score)+(0.2*Evidence Level)+(0.1*FDA Approval), where disease score is 1.0 if exact disease match; 0.9 if “high” match (lobular breast carcinoma is a breast cancer); 0.7 if “medium-high” match (Non clear-cell and clear cell are both Kidney Cancers); 0.5 if “medium” match (All GI system cancers); 0.1 if “low” match (all solid tumors); 0 otherwise (solid vs. heme). Continuing with this example, the evidence level score equals 1.0 if NCCN guidelines; 0.8 if FDA label recommendation; 0.6 if Clinical Research; 0.2 if Case Study in Human; and 0 if Preclinical (e.g. mouse/cell models). Continuing with this example, the FDA Approval score equals 1 if Drug is FDA Approved; and 0 if Drug is unapproved.

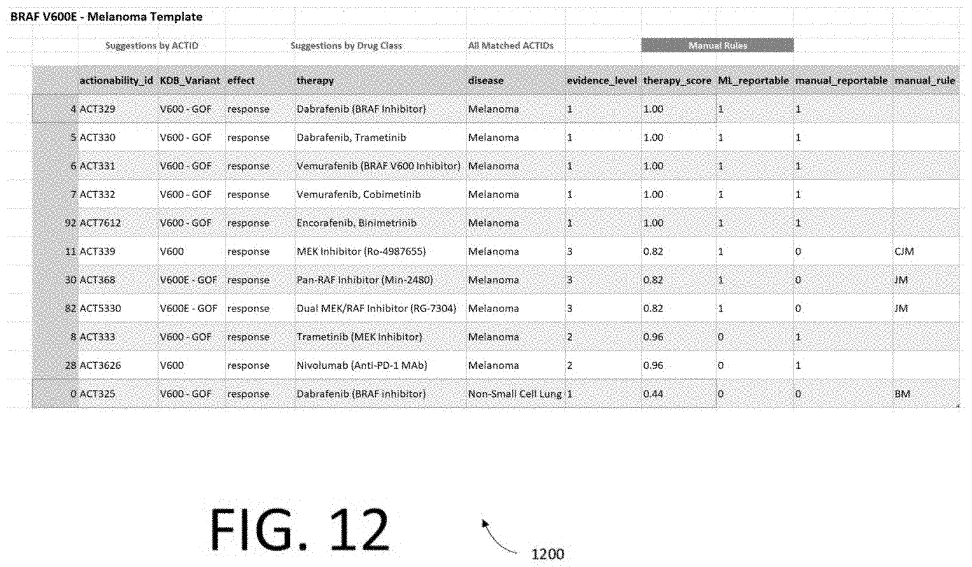

FIG. 12 illustrates a therapy template for a variant and disease state according to an embodiment. Current output of the therapy prioritization engine 140, and system of FIGS. 3a and 3b, for BRAF p.V600E in melanoma. Impact of score therapy highlighted by red boxes, the entry for Dabrafenib in Melanoma is an exact disease match and NCCN level, thus scoring a 1. The entry for Darbafenib in Non-Small Cell Lung Cancer is a solid tumor match as well as NCCN level, but only scores 0.44. Utilizing the scoring system, the most specific entry is prioritized and reported.

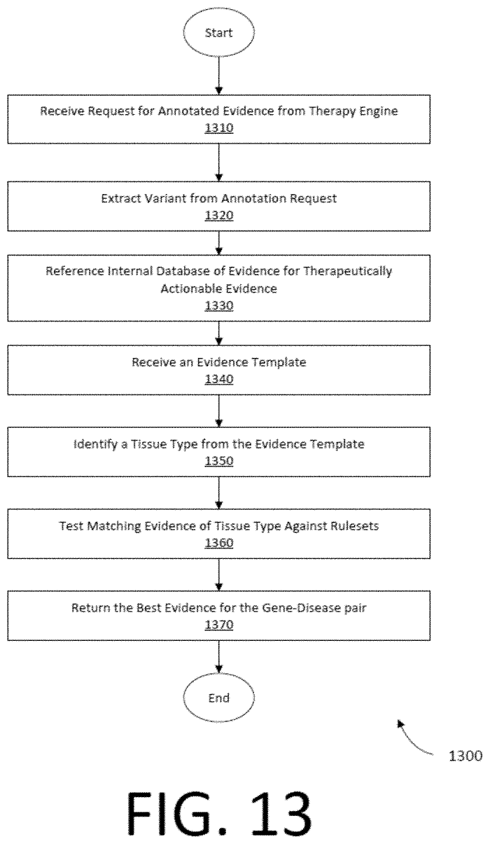

FIG. 13 is a flow diagram 1300 of a process 1300 for receiving a request for annotated evidence. The therapy prioritization engine 140, and system of FIGS. 3a and 3b, “run” is defined as the output by therapy prioritization engine, “gold standard template” is defined as the current set of therapeutic recommendations.

In an example, the therapy prioritization engine may return therapy prioritization information for a PTEN loss-of-function tumor, for example, at receive request for annotated evidence from therapy engine stage 1310. Therapy prioritization engine may then extract variant from annotation request for stage 1320. The engine may then reference an internal database of evidence for therapeutically actionable evidence at stage 1330. In one example, the information may be taken from at least eighty-three different publications abstracted in the internal database. The engine may then receive an evidence template at stage 1340 before identifying a tissue type from the evidence template at stage 1350. The engine may then reference each of the rulesets, such as rule-based selection, AI based selection, and disease specific rules of Therapeutic curation and prioritization module 140 to test matching evidence of tissue type against rulesets at stage 1360. The therapy prioritization engine may return tissue specific evidence for ovarian, breast, glioma, or gastric cancer when prompted for a template with these tissue types at return the best evidence for the gene-disease pair 1370.

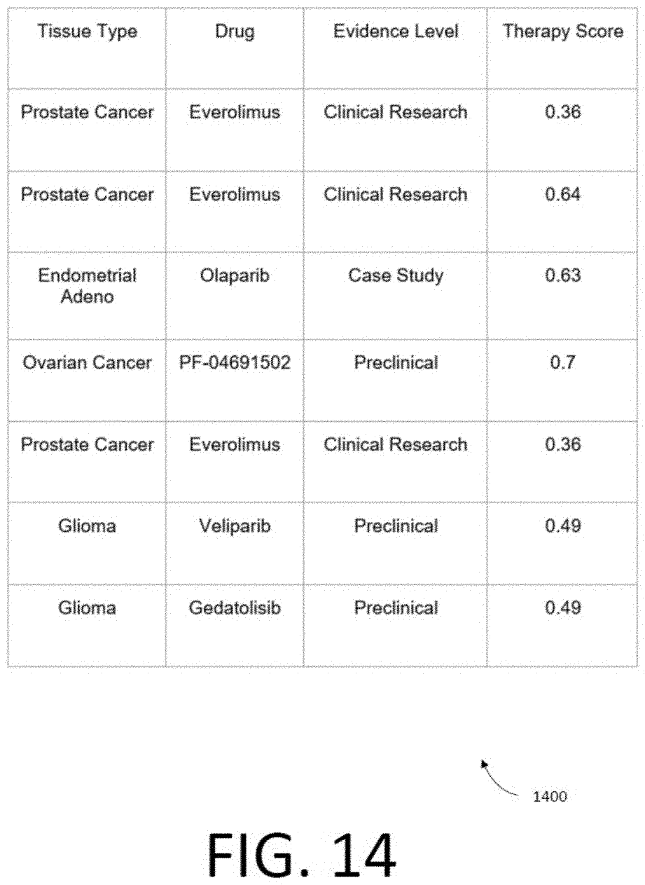

FIG. 14 is a listing of tissue types, example drugs to include on a clinical report, evidence level associated with each respective drug, for the respective tissue type, and a corresponding therapy score, according to an embodiment.

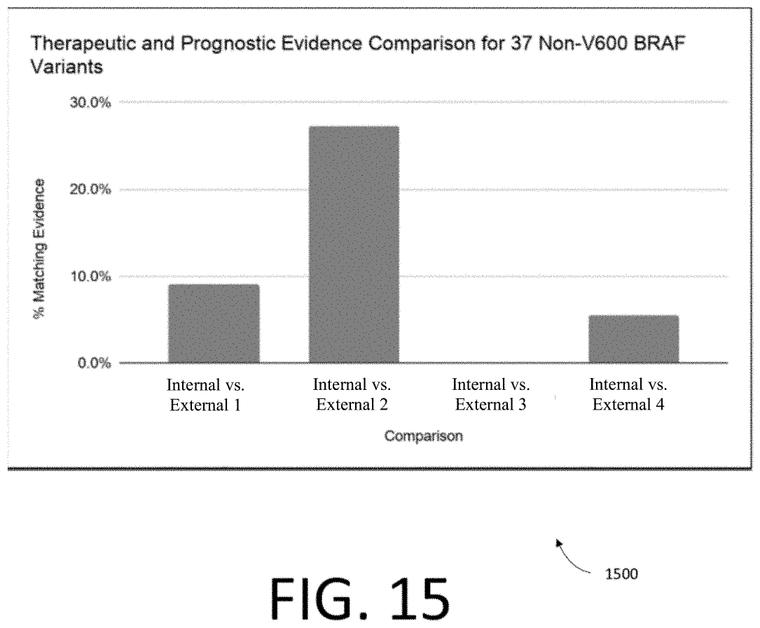

FIG. 15 is a chart comparing the matching evidence between different external databases with an internal database of a laboratory. In one example, the therapeutic and prognostic evidence may be compared for variant/mutation 37 Non-V600 BRAF. In one example, curation may be performed between the internal database and the external database and any matching evidence may be removed for redundancy while other evidence is provided to data-criteria matching module 120 and source article inclusion and exclusion 130 for conversion from the words, terms, concepts, and phrases of the source database to those of the internal database.

Therapy Bypass

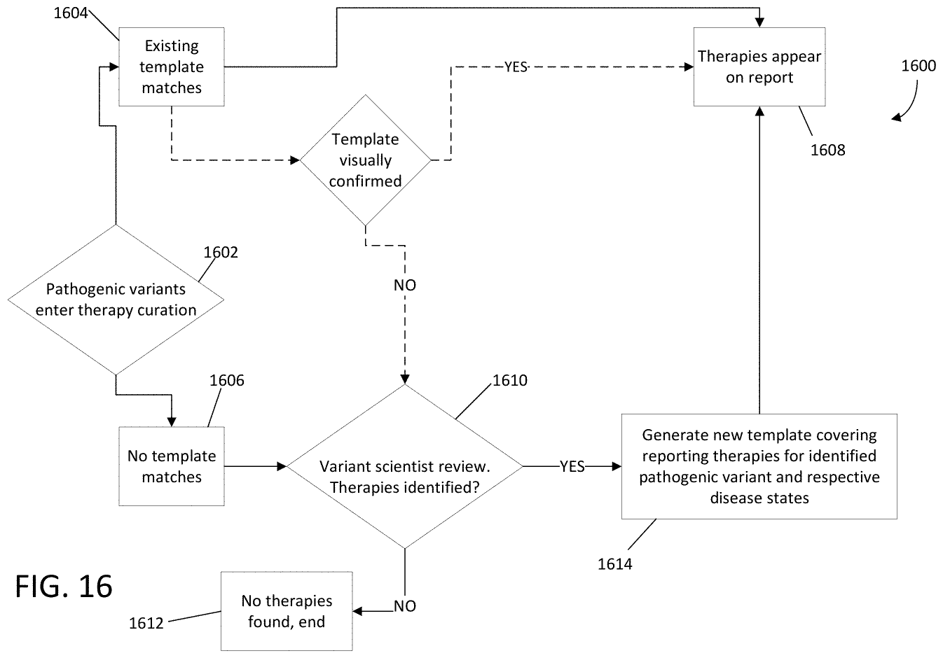

In certain circumstances, new templates may be created or existing templates may be updated or modified as a result of new, highly relevant evidence being ingested into the system. FIG. 16 illustrates one method 1600 by which the system, such as the therapeutic curation and prioritization module 140, may generate a clinical report after curating features at step 1602 from one or more publications and/or from identifying features in one or more sources of clinical information. In this figure, the features are pathogenic variants, although it should be appreciated that the features may be any of the other features discussed herein. After curating the variants, the system determines whether a variant matches existing templates as at step 1604 or whether it has no template match, as at step 1606. Examples of matches may be similarly matches to a disease state ontology, such as an identification of a disease state within the ontology closest in semantic meaning to the disease state, and identification of the closest organ to the disease state, an identification of the most similar disease state based at least in part on genomic similarities, or an identification of the most similar disease state based at least in part on a first cohort of patients having the disease state and a second cohort having the most similar disease state.

With regard to step 1604, the system optionally may determine if the template match can be confirmed manually, e.g., by a user visually comparing the curated variant to the variant(s) listed on each purportedly matching template. If confirmation can be made, or if the optional step is not included, the method then may proceed to include the therapy on a report, such as one of the electronic reports 170, as at step 1608.

If, instead, the user confirmation determines that the variant was inappropriately matched to a template, or if the system did not find any template matches at step 1606, then the method may proceed to step 1610 in which a user may manually review evidence to determine whether he or she can identify one or more potentially relevant therapies. In one embodiment, that evidence may be stored in a knowledge database, such as the evidence store 150. Additionally or alternatively, the evidence may include evidence stored in a non-knowledge database. If no potentially relevant therapies are identified, then no therapies are applied and the method ends with respect to that particular variant, as at step 1612.

If, however, the user identifies one or more potential applicable therapies, as at step 1614, then the user may create a new template matching the identified variant with the identified therapies, so that, through the use of the new template, the identified therapies may appear on the report of step 1612. As intermediary steps, the user identifying the therapies may not have sufficient authority to unilaterally create a new template covering reporting therapies for identified pathogenic variant and respective disease states, as at step 1614. For example, the user may propose a new template matching the identified variant to the identified one or more therapies, to one or more individuals with authority to sign off on the proposed template. Then, after refreshing the templates to confirm that this new template is included in the data storage of available templates, the new template may be applied to cause the identified therapies to apply on the report of step 1612.

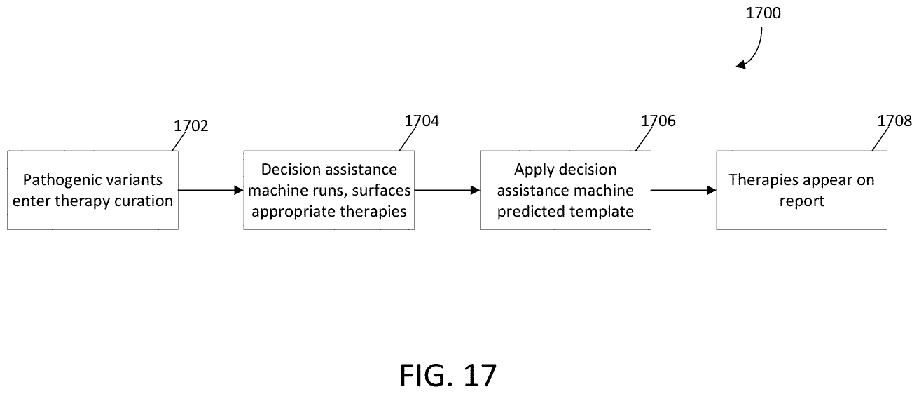

In another embodiment, the therapy prioritization engine 100 may include a bypass feature permitting an analysis to proceed directly from a variant or other feature analysis to report generation and/or trial matching, without engaging in a therapy curation step, for example, within therapeutic curation module 140 and/or a step of human review or sign-off of the curated therapies. In this embodiment, as represented by FIG. 17, an alternative method 1700 by which the system may generate a clinical report after curating features at step 1702 from one or more publications and/or from identifying features in one or more sources of clinical information. As with the previous example, the features in this figure are pathogenic variants, although it should be appreciated that the features may be any of the other features discussed herein. In this example, after curating the variants, the system bypasses the template matching steps of the example of FIG. 16. Instead, the decision assistance machine may run, identifying appropriate therapies by itself, as at step 1704, and selecting one or more machine predicted templates that include the identified therapies, at step 1706, with the end result of the therapies appearing on a report, at step 1708.

Alternatively, the bypass feature may entirely skip processes related to therapy curation to instead identify one or more trials for which the patient may qualify. This bypass may be of more significance when there are no established therapies for patients that sufficiently match the features of the reference patient being analyzed, although it should not be limited to just those circumstances.

For the decision assistance machine to identify appropriate therapies or trials, it may employ an artificial intelligence engine using a plurality of rule sets, machine learning models, and/or neural networks to deliver potential therapeutic matches to patients, e.g., based on matches to multiple features identified in one or more sources of patient-related clinical information with features curated from one or more publications and/or data stored within the knowledge database. As noted above, the therapy prioritization engine of the decision assistance machine may include a sophisticated hierarchical disease matching algorithm, variant-specific logic to identify potential therapies, and an explicit rules engine to identify the potential therapeutic matches.

For example, the data assistance machine may match one or more of cancer cohort, diagnosis, age, mutated gene name/variants, microsatellite instability presence and/or status, pertinent negatives, and/or tumor mutation burden values or ranges and assign a bypass when one or more of those criteria match structured elements within a patient's data. For example, the system may bypass template review for specific variants or biomarkers relating to one or more specific disease states. Each feature being matched also may include one or more sub-features to provide even more granularity to the match. For example, within variants, the data assistance machine may match single nucleotide variations (SNVs), indels, germline data, copy number variations (CNVs), fusions, isoforms, and/or RNA expressions. In one example, the data assistance machine may use a gene name, variant type (including one or more of SNV/indel, CNV, fusion, or RNA expression information), mutation information (including one or more of p./c., copy number loss and/or gain, and chromosomal rearrangement), and cancer type to create suggested therapies using the latest reported evidence. “Matches” may be qualified using one or more of the heuristics discussed above. For example, a multiple variant match to a particular patient may result in a particular therapy being deemed a more significant match or reported above other therapies if the multiple variants are part of an additive heuristic, or a first therapy may be reported above a second therapy if the combination of variants triggers a replacement heuristic in which the first therapy is seen as being more effective or otherwise notable.

In this embodiment, the system may find direct or indirect matches between the clinical information and the publications or knowledge database information. In the event of direct matches, i.e., where the patient information perfectly matches relevant publication and/or KDB information, the data assistance machine may be able to identify relevant therapies and/or trials automatically. Conversely, when only indirect, i.e., partial, matches are possible, the data assistance machine still may be able to identify relevant therapies and/or trials based on a number and closeness of match of features. The system also may incorporate manual review to confirm those indirect matches, as well as to identify matches that the machine is unable to make.

In some aspects, the system may be able to retrieve the features automatically from the clinical information and/or knowledge database. Alternatively, the system may not be able to obtain certain features, such as disease type, with sufficient confidence so as to curate them automatically. In such situations, the system may include a user interface having an input selector enabling a user to manually select those features. That input selector may include a user-selectable list, a drop-down menu of possible choices, a text entry box, or another type of input as would be appreciated by those of ordinary skill in the relevant art. In still another aspect, the system may require manual review even if the system is able to identify the necessary patient information or match that to therapy information stored in the knowledge database.

In order to determine whether automatic or manual review may be carried out, the data assistance machine may apply a rule set after analyzing the curated data. For example, if the machine output does not contain any therapies or if the patient data does not include any relevant biomarkers, the system may trigger a therapy bypass to send the case straight to a trial matching phase or to a template designed for such situations.

Alternatively, if the data assistance machine returns an error message, the system may trigger a manual review, e.g., to send the case to a therapy curation phase. Manual review also may be triggered if the machine produces the same therapy matching to multiple variants and if an effect field for different entries contains both resistance and response effect field entries.

If the patient's sequencing results return one or more relevant biomarkers or fusions and one or more potential treatments, the system then may analyze those biomarkers and/or other structured elements within the patient's data to determine if the patient is a member of one or more cohorts designated as bypass cohorts. One such example of a structured element may be the patient's disease state, and exemplary disease states that may correspond to bypass scenarios are listed in the paragraphs that follow.

Another example may be if the patient possesses one or more specific biomarkers or variants, also as discussed below, or one or more specific structured elements within the patient's molecular data. For example, the system may bypass review and produce a templated report if the patient's sequenced results return one or more combinations of hormone receptors, alone or in combination with particular disease states. In one non-limiting example, the system may have a template indicating that review is not necessary if the reported therapies are non-hormonal and if the patient's sequencing results test negative for hormone receptors known to correlate with the patient's disease state. Examples of such biomarker-related data may by the presence or absence, generally, or the presence or absence of specific biomarkers such as SNVs, Indels, CNVs, MSI, TMB, existence of the variant in the patient's germline sample, existence of the variant in the patient's somatic sample, fusion pairs, single gene fusions, specific variants, and/or specific self-fusions.

In still another example, alone or in combination with one or more of the other factors discussed herein, the approval status of reported treatments may serve as a bypass trigger. For example, if one or all of the therapies to be reported reflect on label treatments for the patient's disease state, the system may then trigger the bypass to report such treatments without requiring manual review.

It will be understood that bypass may be triggered when one of these criteria is met or, alternatively, when a combination of criteria are met. As to the latter case, for example, the system may determine that the patient is bypass-eligible based on the patient's extracted disease state, evaluate whether the patient's relevant biomarkers match bypass-eligible biomarkers, and then evaluate the reported therapies to determine whether they are on or off-label, with bypass being triggered when one or all of the identified therapies are determined to be on label for the patient's disease state.

Still further, the system may trigger a manual review if the cohort or disease used as input matches but the machine output contains a therapy on a blacklist. In particular, while the system may designate one or more therapies as sufficiently well-established so as to be whitelisted and reportable without additional review and/or sign-off, at the other end of the spectrum, one or more other therapies may be associated with at least a threshold degree of confidence that they do not apply to the matched cohort or disease. In those situations, although the therapy may be blacklisted with regard to the cohort or disease match, the system still may trigger manual review to confirm its inapplicability prior to excluding it from reporting. Some examples of this last use case may include a recommendation for manual review when the machine returns pembrolizumab as a therapy while also recognizing that the patient has one of the following: Gastric Cancer (PD-L1 positive AND CPS >=1); Cervical Cancer (PD-L1 positive AND CPS >=1); Triple-Receptor Negative Breast Cancer (PD-L1 positive AND CPS >=1); Breast Cancer (PD-L1 positive AND CPS >=1); Esophageal Cancer (PD-L1 positive AND CPS >=1); Esophageal Adenocarcinoma (PD-L1 positive AND CPS >=1); or Esophageal Squamous Cell Carcinoma (PD-L1 positive AND CPS >=1). Similarly, the system may trigger manual review if the recommended therapy is venetoclax for a patient with chronic lymphocytic leukemia (17p deletion), capmatinib or tepotinib for non-small cell lung cancer (MET exon 14 skipping), or pemetrexed for non-small cell lung cancer (NOT squamous cell). It will be appreciated that, in some embodiments, not all of these treatments may end up as part of an implemented blacklist and/or that a blacklist may include treatments other than those listed here. In one instance, such therapies may exceed a first threshold below which the system has determined that they can be blacklisted without additional review, e.g., when the therapy has been contraindicated for the particular cohort or disease, but fail to surpass a second threshold above which therapies would not be considered blacklisted.

In one embodiment, therapies or combinations of therapies with certain disease states may be treated as “whitelisted” by default, so that if they do not appear on a manual review-triggering blacklist, the system will trigger the therapy bypass. Alternatively, the system may include a formal whitelist of therapies and/or therapy/disease state combinations that trigger the therapy bypass, in addition to a formal blacklist of therapies and/or therapy/disease state combinations that trigger a manual review. In the latter instance, therapies on neither the whitelist nor the blacklist may be evaluated according to the other rules of the ruleset.

The whitelist of therapies may correlate disease states with one or more of publications, therapies, and features such as variants. For example, one entry in a whitelist may correlate the mesenchymal cell neoplasm class of tissue tumors with a particular journal article discussing the specific use of the therapy trastuzumab in connection with chemotherapy to treat metastatic breast cancer in patients with HER2 overexpression.

Potential disease states may include a generic cancer state or specific disease states, where the specific disease states may include, e.g., blastomas, carcinomas, leukemias, lymphomas, melanomas, sarcomas, etc. The disease states also may include categories such as childhood cancers, chronic cancers, or congenital cancers. Still further, the disease states may include organ-related cancers, such as brain, breast, colon/colorectal, lung, etc. Specifically, the disease states may include but not be limited to one or more of: Acral Lentiginous Melanoma, Acute Leukemia, Acute Lymphoblastic Leukemia, Acute Myeloid Leukemia, Acute Promyelocytic Leukemia, Adenoid Cystic Carcinoma, Adrenal Cortex Neoplasm, Adrenocortical Carcinoma, Adult Acute Lymphoblastic Leukemia, Adult B Acute Lymphoblastic Leukemia, Adult T-Cell Leukemia/Lymphoma, Alveolar Rhabdomyosarcoma, Alveolar Soft Part Sarcoma, Ameloblastoma, Anaplastic Astrocytoma, Anaplastic Large Cell Lymphoma, Anaplastic Oligoastrocytoma, Anaplastic Oligodendroglioma, Anaplastic Pleomorphic Xanthoastrocytoma, Angiomatoid Fibrous Histiocytoma, Angiosarcoma, Astroblastoma, Astrocytic Tumor, Astrocytoma, Atypical Spitz Nevus, B Acute Lymphoblastic Leukemia, Basal Cell Carcinoma, B-Cell Non-Hodgkin Lymphoma, Bile Duct Cancer, Bladder Cancer, Bladder Urothelial Carcinoma, Bone Marrow Cancer, Brain Cancer, Brain Glioblastoma, Breast Cancer, Bronchiolo-Alveolar Adenocarcinoma, Burkitt Lymphoma, Carcinoma, Castration-Resistant Prostate Carcinoma, Central Nervous System Hemangioblastoma, Central Nervous System Lymphoma, Central Nervous System Neoplasm, Cervical Adenocarcinoma, Cervical Cancer, Childhood Acute Lymphoblastic Leukemia, Childhood B Acute Lymphoblastic Leukemia, Childhood Glioblastoma, Childhood Leukemia, Childhood Neuroblastoma, Childhood Rhabdomyosarcoma, Cholangiocarcinoma, Chordoma, Chronic Leukemia, Chronic Lymphocytic Leukemia, Chronic Myeloid Leukemia, Chronic Myelomonocytic Leukemia, Chronic Myeloproliferative Disease, Chronic Neutrophilic Leukemia, Clear Cell Sarcoma, Colon Cancer, Colon Mucinous Adenocarcinoma, Colorectal Adenocarcinoma, Colorectal Cancer, Congenital Fibrosarcoma, Congenital Peribronchial Myofibroblastic Tumor, Cutaneous Melanoma, Dermatofibrosarcoma Protuberans, Desmoid-Type Fibromatosis, Desmoplastic Small Round Cell Tumor, Diffuse Astrocytoma, Diffuse Gastric Adenocarcinoma, Diffuse Intrinsic Pontine Glioma, Diffuse Large B-Cell Lymphoma, Diffuse Large B-Cell Lymphoma Activated B-Cell Type, Ductal Breast Carcinoma, Ductal Breast Carcinoma In Situ, Eccrine Porocarcinoma, Endometrial Adenocarcinoma, Endometrial Cancer, Endometrial Stromal Sarcoma, Endometrioid Adenocarcinoma, Endometrioid Ovary Carcinoma, Endometrioid Tumor, Ependymoma, Epithelioid Hemangioendothelioma, ER+ Breast Cancer, Erdheim-Chester Disease, Esophageal Adenocarcinoma, Esophageal Cancer, Esophageal Squamous Cell Carcinoma, Essential Thrombocythemia, Ewing Sarcoma, Extranodal Marginal Zone Lymphoma of Mucosa-Associated Lymphoid Tissue, Extraskeletal Myxoid Chondrosarcoma, Fibrous Histiocytoma, Follicular Lymphoma, Gallbladder Cancer, Ganglioglioma, Gastric Adenocarcinoma, Gastric Adenosquamous Carcinoma, Gastric Cancer, Gastroesophageal Junction Adenocarcinoma, Gastrointestinal Neuroendocrine Tumor, Germ Cell Tumor, Glioblastoma, Glioma, Glomus Tumor, Hairy Cell Leukemia, Head and Neck Cancer, Head and Neck Squamous Cell Carcinoma, Hematopoietic and Lymphoid Cell Neoplasm, Hepatocellular Carcinoma, Hepatocellular Fibrolamellar Carcinoma, Her2− Breast Cancer, Her2+ Breast Cancer, Hereditary Diffuse Gastric Adenocarcinoma, Hidradenocarcinoma, Hidradenoma, High Grade B-Cell Lymphoma with MYC and BCL2 or BCL6 Rearrangements, High Grade Ovarian Serous Adenocarcinoma, Histiocytic Sarcoma, HR− Breast Cancer, HR+ Breast Cancer, HR+ Her2− Breast Cancer, Human Papillomavirus Positive Oropharyngeal Squamous Cell Carcinoma, Hypereosinophilic Syndrome, Inflammatory Myofibroblastic Tumor, Intrahepatic Cholangiocarcinoma, Invasive Bladder Transitional Cell Carcinoma, Invasive Breast Carcinoma, Invasive Lobular Carcinoma, Kidney Cancer, Langerhans Cell Histiocytosis, Laryngeal Squamous Cell Carcinoma, Larynx Cancer, Leiomyosarcoma, Leukemia, Lipoblastoma, Liposarcoma, Liver Cancer, Low Grade Glioma, Luminal A Breast Carcinoma, Lung Acinar Adenocarcinoma, Lung Adenocarcinoma, Lung Cancer, Lung Mucoepidermoid Carcinoma, Lung Neoplasm, Lung Squamous Cell Carcinoma, Lymphangioleiomyomatosis, Lymphoma, Major Salivary Gland Carcinoma ex Pleomorphic Adenoma, Malignant Anus Melanoma, Malignant Glioma, Malignant Mesothelioma, Malignant Peripheral Nerve Sheath Tumor, Malignant Pleural Mesothelioma, Malignant Soft Tissue Neoplasm, Mammary Analog Secretory Carcinoma of Salivary Gland, Mantle Cell Lymphoma, Medulloblastoma, Megakaryocytic Leukemia, Melanocytoma, Melanoma, Meningioma, Merkel Cell Carcinoma, Mesenchymal Cell Neoplasm, Mesenchymal Chondrosarcoma, Mesothelioma, Metastatic Colorectal Carcinoma, Metastatic Cutaneous Melanoma, Metastatic Endometrial Carcinoma, Metastatic Melanoma, Metastatic Urothelial Carcinoma, Micropapillary Lung Adenocarcinoma, MiT Family Translocation-Associated Renal Cell Carcinoma, Mucoepidermoid Carcinoma, Mucosal Melanoma, Multiple Myeloma, Myelodysplastic Myeloproliferative Cancer, Myelodysplastic Syndrome, Myelofibrosis, Myeloid Neoplasm, Myeloid/Lymphoid Neoplasms with Eosinophilia and Rearrangement of PDGFRA, PDGFRB, or FGFR1, or with PCM1-JAK2, Myeloid/Lymphoid Neoplasms with FGFR1 Rearrangement, Myofibromatosis, Myxoid Liposarcoma, Nasal Type Extranodal NK/T-Cell Lymphoma, Nasopharynx Carcinoma, Neuroblastoma, Neuroendocrine Tumor, Neuronal and Mixed Neuronal-Glial Tumors, Non-Hodgkin Lymphoma, Non-Small Cell Lung Cancer, NUT Carcinoma, Olfactory Groove Meningioma, Oligodendroglioma, Oral Squamous Cell Carcinoma, Oropharyngeal Squamous Cell Carcinoma, Oropharynx Cancer, Ossifying Fibromyxoid Tumor, Osteosarcoma, Ovarian Adenocarcinoma, Ovarian Cancer, Ovarian Clear Cell Carcinoma, Ovarian Serous Carcinoma, Ovary Epithelial Cancer, Paget Disease of the Scrotum, Pancreas Adenocarcinoma, Pancreatic Cancer, Pancreatic Ductal Carcinoma, Pancreatic Endocrine Carcinoma, Pancreatic Neuroendocrine Tumor, Papillary Adenocarcinoma, Papillary Craniopharyngioma, Papillary Renal Cell Carcinoma, Papillary Thyroid Carcinoma, PEComa, Pediatric Low-Grade Glioma, Pharyngeal Squamous Cell Carcinoma, Philadelphia Chromosome Negative, BCR-ABL1 Positive Chronic Myelogenous Leukemia, Pilocytic Astrocytoma, Pleomorphic Xanthoastrocytoma, Pleural Mesothelioma, Polycythemia Vera, Precursor Lymphoid Neoplasm, Primary Cutaneous T-Cell Non-Hodgkin Lymphoma, Primary Myelofibrosis, Prostate Cancer, Prostate Neuroendocrine Neoplasm, Pseudomyogenic Hemangioendothelioma, Recurrent Glioblastoma, Recurrent Ovarian Carcinoma, Renal Cell Carcinoma, Renal Clear Cell Carcinoma, Retinoblastoma, Rhabdoid Cancer, Rhabdomyosarcoma, Rosai-Dorfman Disease, Salivary Gland Adenocarcinoma, Salivary Gland Adenoid Cystic Carcinoma, Salivary Gland Carcinoma, Salivary Gland Myoepithelial Carcinoma, Salivary Gland Neoplasm, Sarcoma, Schwannoma, Sclerosing Epithelioid Fibrosarcoma, Sezary Syndrome, Skin Squamous Cell Carcinoma, Small Cell Carcinoma, Small Cell Lung Cancer, Soft Tissue Sarcoma, Solid Tumors, Solitary Fibrous Tumors, Sporadic Breast Cancer, Squamous Cell Carcinoma, Squamous Cell Carcinoma of the Penis, Synovial Sarcoma, Systemic Mastocytosis, Systemic Mastocytosis with an Associated Hematological Neoplasm, T Acute Lymphoblastic Leukemia, T-Cell and NK-Cell Neoplasm, Tenosynovial Giant Cell Tumor, Thymic Carcinoma, Thymic Squamous Cell Carcinoma, Thymus Cancer, Thyroid Cancer, Thyroid Gland Anaplastic Carcinoma, Thyroid Gland Hyalinizing Trabecular Tumor, Thyroid Hurthle Cell Carcinoma, Thyroid Medullary Carcinoma, Triple-Receptor Negative Breast Cancer, Urothelial Carcinoma, Uterine Corpus Endometrial Carcinoma, Uterine Corpus High Grade Endometrial Stromal Sarcoma, Uterine Corpus Myxoid Leiomyosarcoma, Uterine Corpus Serous Adenocarcinoma, Uterus Leiomyosarcoma, Uveal Melanoma, Vulvar Carcinoma, Waldenstrom Macroglobulinemia, or Wilms Tumor.

The whitelist may classify the type of relationship between the therapy and/or variant and the disease state. For example, a whitelist may determine that those entities can be related either as “diagnostic,” “prognostic,” or “therapeutic.” For entities that are related as “diagnostic,” the whitelist may classify them further if the system is able to determine the type of relationship between them. In particular, the system may further classify diagnostic relationships as “associated,” “diagnostic,” or “NA for evidence type.” “Associated” may mean that a certain variant is common in the disease with which it is associated, although that disease is not necessarily defined by the variant. For example, a CDH1 variant may be “associated” with breast cancer even though breast cancer is not defined by the presence of a CDH1 mutation. Conversely, “diagnostic” may refer to a situation where the disease is defined by the presence of the variant. For example, chronic myeloid leukemia (CML) is defined by BCR-ABL1 fusions, so that the relationship between CML and BCR-AML1 is “diagnostic.” For entities that are related as “prognostic,” the whitelist may classify them further in terms of an “equivalent prognosis,” a “favorable prognosis,” a “favorable risk,” an “increased risk,” an “intermediate risk,” a “poor risk,” or an “unfavorable prognosis.” For entities that are related as therapeutic, the whitelist further may classify them as “conflicting evidence,” “neutral,” “non-response,” “reduced response,” “resistance,” or “response.” Additionally or alternatively, the whitelist may classify entries according to a variant type, e.g., as a biomarker, copy number variant, expression, fusion, protein functional, protein positional, transcript positional, or variant group.

The whitelist further may relate a therapy directly to one or more particular variants. Additionally, the whitelist may cross-correlate a therapy to one or more of the categories of components other than the variant(s) to which it relates. For example, a particular therapy such as “imatinib” may be related to multiple genomic types such as fusion, protein positional, and protein functional, multiple disease states such as Dermatofibrosarcoma Protuberans, Acute Myeloid Leukemia, Chronic Myeloid Leukemia, and Hypereosinophilic Syndrome, and multiple publications. From these cross-categorizations, the system may be able to determine whether a particular therapy can be whitelisted without access to a patient's particular variant information or, if that variant information is available, to determine whether the therapy can be whitelisted in view of the other information that is available besides the patient's variant information, e.g., based solely on the patient's disease type and/or an authoritativeness of the publication discussing the therapy.

The rule set may also include a rule indicating that therapy bypass may be triggered if the data assistance machine identifies multiple interacting therapies related to the patient features being analyzed.

If none of the rules above apply, then the system may trigger the therapy bypass as a default option rather than sending the case to manual review.

The following may exemplify one set of bypass rules related to the data assistance machine:

First, determine if the patient's records establish that it is a member of a cohort of patients having at least one feature in common. In this case, the feature may be a disease state and may be selected from among: Acute Lymphocytic Leukemia, Acute Myeloid Leukemia, Adrenal Cancer, Basal Cell Carcinoma, Cervical Cancer, Chromophobe Renal Cell Carcinoma, Cervical Cancer, Chronic Myeloid Leukemia, Clear Cell Renal Cell Carcinoma, Colorectal Cancer, Endometrial Cancer, Gastric Cancer, Gastrointestinal Stromal Tumor, Glioblastoma, Hairy Cell Leukemia, Head and Neck Squamous Cell Carcinoma, Liver Cancer, Medulloblastoma, Megakaryoblastic Leukemia, Melanoma, Meningioma, Mesothelioma, Multiple Myeloma, Neuroblastoma, Oropharyngeal Cancer, Ovarian Cancer, Pancreatic Cancer, Peritoneal Cancer, Prostate Cancer, Retinoblastoma, Skin Cancer, Small Cell Lung Cancer, T Cell Lymphoma, Testicular Cancer, Thymoma, Tumor of Unknown Origin, or Uveal Melanoma. If, during the course of analyzing the patient's clinical records to determine disease state information, the system encounters a disease state with either “sarco” or “neuroendocrine” in the path diagnosis, the bypass analysis may terminate and the case will be sent to a manual review workflow so that a user can manually select the disease type.

Second, analyze the patient clinical information to determine if it is associated with a complete programmed death-ligand 1 (“PD-L1”) or DNA mismatch repair (“MMR”) report.

If the information is related to a complete PD-L1 SP142 IHC report, then the system may grab the results and a CPS score. If the result is positive and based on CPS score, then the drug pembrolizumab may be considered on label, whereas if the result is negative or based on CPS score, then pembrolizumab may be considered off label. If the information is related to a complete PD-L1 22C3 IHC report, then the system may grab the results. If the result is positive, then the drug atezolizumab may be considered on label, whereas if the result is negative, then atezolizumab may be considered off label. If the information is related to a complete PD-L1 28-8 IHC report, then the system may grab the results. If the result is positive, then the drug nivolumab may be considered on label, whereas if the result is negative, then nivolumab may be considered off label.

Similarly, if the information relates to an MMR report and if the MMR report is complete, the system may obtain the results. If the MMR result is dMMR, then the drug dostarlimab-gxly may be considered on label, whereas if the MMR result is pMMR, then dostarlimab-gxly may be considered off label.

Once those curation steps have been performed, the data assistance machine may be fed one or more of the following inputs derived from patient clinical data: SNVS, INDELS, CNVS, MSI, TMB, Germline, Fusion pairs, Single gene fusions, Cohort, EGFR self fusions, PD-L1, or MMR.

If, instead, the patient's records establish that it is not a member of one of the cohorts discussed above, then a user needs to manually specify the disease type. For example, the user may be prompted to select from a defined list of diseases. This list of diseases already may be mapped in the knowledge database to one or more therapies, trials, variants, etc.

The system then may determine if the patient information contains a reportable MET Exon 14 Skipping variant. As part of that process, if the system determines that SNV/Indel occurs between c. position A and B in MET, then the system will designate the patient record for manual review to determine if a Met Exon 14 variant is present, as the MET 14 Exon Skipping Variant is needed for on/off labeling. Additionally or alternatively, the system may check for PD-L1 and MMR for the patient's most recent results, with the same rules discussed above for whether either is present applying similarly here.

As with the other situation just discussed, once these curation steps have been performed, the data assistance machine may be fed one or more of the following inputs: SNVS, INDELS, CNVS, MSI, TMB, Germline, Fusion pairs, Single gene fusions, Cohort, EGFR self fusions, PD-L1, or MMR.

In this situation, once the data assistance machine analyzes the patient information, the following rules may be applied. First, the data assistance machine may determine whether the drug therapy venetoclax or ibrutinib is matched. If so, the patient record may be designated for manual review for 17p data. Specifically, if a reviewer determines that 17p is present, then venetoclax or ibrutinib can be designated as on label. Conversely, if 17p is not present, then venetoclax or ibrutinib can be designated as off label. The distinction between on label and off label-designated therapies may factor into a reporting phase. For example, the report that is generated, either via a template or when templates are bypassed, may include a first section designating on label therapies and a second label designating off label therapies, or the report may be sortable by the user according to on/off label status.

Whether a template bypass is applied or not, the reporting template may have restrictions on the number of treatments or other links that it can present to the user. Such restrictions may occur, e.g., as a result of space constraints on a display screen. For example, the system may format the reporting template for display on the screen of a computing device (a computer monitor, laptop screen, tablet, mobile device screen, etc.) and only present links that can be displayed concurrently on the screen. Thus, the hierarchical rule set may include this screen size and/or resolution as one of the criteria to be evaluated when ranking and/or determining to exclude one or more publications or the disease states reported therein.

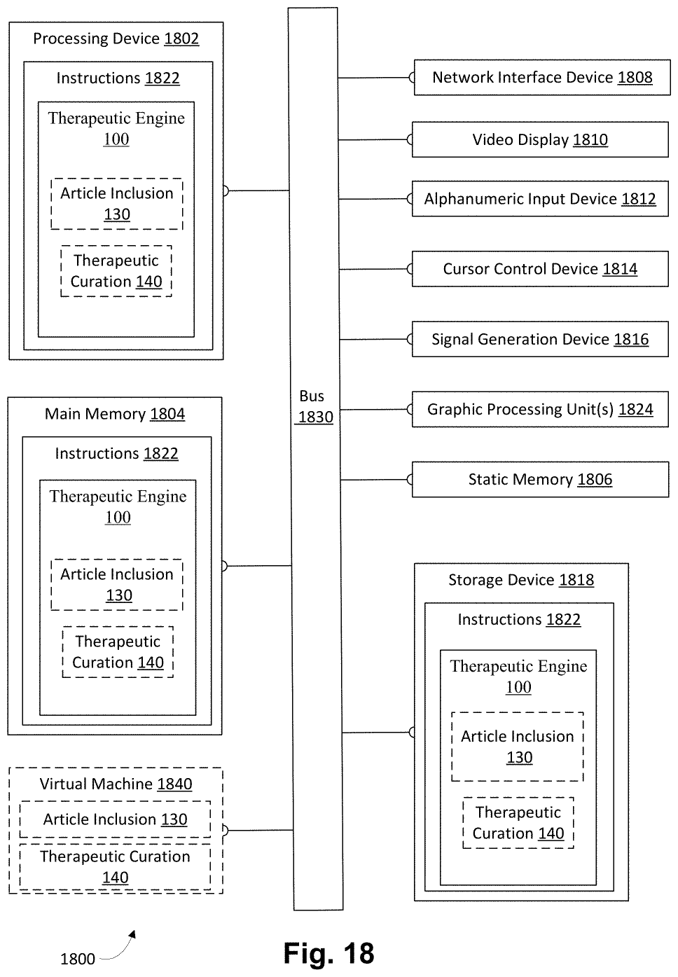

FIG. 18 is an illustration of an example machine of a computer system 1800 within which a set of instructions, for causing the machine to perform any one or more of the methodologies discussed herein, may be executed. In alternative implementations, the machine may be connected (such as networked) to other machines in a LAN, an intranet, an extranet, and/or the Internet.

The machine may operate in the capacity of a server or a client machine in client-server network environment, as a peer machine in a peer-to-peer (or distributed) network environment, or as a server or a client machine in a cloud computing infrastructure or environment. The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 1800 includes a processing device 1802, a main memory 1804 (such as read-only memory (ROM), flash memory, dynamic random access memory (DRAM) such as synchronous DRAM (SDRAM) or DRAM, etc.), a static memory 1806 (such as flash memory, static random access memory (SRAM), etc.), and a data storage device 1818, which communicate with each other via a bus 1830.

Processing device 1802 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 1802 may also be one or more special-purpose processing devices such as an application specific integrated circuit (ASIC), a field programmable gate array (FPGA), a digital signal processor (DSP), network processor, or the like. The processing device 1802 is configured to execute instructions 1822 for performing the operations and steps discussed herein.

The computer system 1800 may further include a network interface device 1808 for connecting to the LAN, intranet, internet, and/or the extranet. The computer system 1800 also may include a video display unit 1810 (such as a liquid crystal display (LCD) or a cathode ray tube (CRT)), an alphanumeric input device 1812 (such as a keyboard), a cursor control device 1814 (such as a mouse), a signal generation device 1816 (such as a speaker), and a graphic processing unit 1824 (such as a graphics card).

The data storage device 1818 may be a machine-readable storage medium 1828 (also known as a computer-readable medium) on which is stored one or more sets of instructions or software 1822 embodying any one or more of the methodologies or functions described herein. The instructions 1822 may also reside, completely or at least partially, within the main memory 1804 and/or within the processing device 1802 during execution thereof by the computer system 1800, the main memory 1804 and the processing device 1802 also constituting machine-readable storage media.

In one implementation, the instructions 1822 include instructions for a Therapeutic Engine (such as the Therapeutic Engine 100 of FIG. 1) and/or a software library containing methods that function as a Therapeutic Engine. The instructions 18622 may further include instructions for an Article inclusion 130, such as Source Article Inclusion & Exclusion 130 and Therapeutic Curation 140, such as Therapeutic Curation and Prioritization 140 of FIG. 1. While the machine-readable storage medium 1828 is shown in an example implementation to be a single medium, the term “machine-readable storage medium” should be taken to include a single medium or multiple media (such as a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media. The term “machine-readable storage medium” shall accordingly exclude transitory storage mediums such as signals unless otherwise specified by identifying the machine-readable storage medium as a transitory storage medium or transitory machine-readable storage medium.

In another implementation, a virtual machine 1840 may include a module for executing instructions for an Article inclusion 130, such as Source Article Inclusion & Exclusion 130 and Therapeutic Curation 140, such as Therapeutic Curation and Prioritization 140 of FIG. 1. In computing, a virtual machine (VM) is an emulation of a computer system. Virtual machines are based on computer architectures and provide functionality of a physical computer. Their implementations may involve specialized hardware, software, or a combination of hardware and software.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “providing” or “calculating” or “determining” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various general purpose systems may be used with programs in accordance with the teachings herein, or it may prove convenient to construct a more specialized apparatus to perform the method. The structure for a variety of these systems will appear as set forth in the description below. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (such as a computer). For example, a machine-readable (such as computer-readable) medium includes a machine (such as a computer) readable storage medium such as a read only memory (“ROM”), random access memory (“RAM”), magnetic disk storage media, optical storage media, flash memory devices, etc.

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

1. A method for associating published media with a subject, comprising:
receiving a subject specimen comprising a cancerous biological specimen;
sequencing the specimen to obtain subject genomic data, the subject genomic data comprising a first plurality of at least 10,000 sequence reads, in electronic form, of an RNA sample comprising RNA molecules from the cancerous biological specimen;
identifying a first alteration nomenclature match to the subject genomic data in first genomic data extracted from a first published medium, the first published medium also including a first disease state, a first treatment, and a first outcome, the first genomic data including at least a first pattern of gene expression and a corresponding first genomic type, the first treatment associated with the first outcome when treating the first disease state expressing the at least first pattern of gene expression;
identifying a second alteration nomenclature match to the subject genomic data in second genomic data extracted from a second published medium, the second published medium also including the first disease state, a second treatment, and a second outcome, the second genomic data including at least a second pattern of gene expression and a corresponding second genomic type, the second treatment associated with the second outcome when treating the first disease state expressing the at least second pattern of gene expression;
applying a hierarchical rule set to the first and second published media based at least in part on the first and second alteration nomenclature matches and one or more evidence metrics, the hierarchical rule set determining to report the first treatment and to exclude reporting of the second treatment despite the second published medium including at least one match between its extracted disease state and a subject disease state;
identifying a reporting template based at least in part on the subject genomic data and the subject disease state;
generating a report using the identified reporting template, the report reporting treatments according to the hierarchical rule set;
comparing the report to one or more approval criteria; and
publishing the report when the approval criteria are satisfied.

2. The method of claim 1, wherein the subject genomic data includes both germline and somatic data.

3. The method of claim 1, wherein the published media comprises one or more of written media, video media, audio media, or audio/visual media.

4. The method of claim 1, wherein the subject disease state is cancer, cardiology, depression, mental health, diabetes, infectious disease, epilepsy, dermatology, or autoimmune disease.

5. The method of claim 1, wherein the subject disease state is cancer and wherein the first treatment or the second treatment is one of: surgery, chemotherapy, radiation therapy, bone marrow transplant, immunotherapy, hormone therapy, targeted drug therapy, cryoablation, radiofrequency ablation, a medication, or a clinical trial.

6. The method of claim 1, wherein the first outcome or the second outcome is a measurable change in health, function, or quality of life.

7. The method of claim 1, wherein the first outcome or the second outcome is a prognosis or side effect.

8. The method of claim 1, wherein the first pattern of gene expression or the second pattern of gene expression is a sequence of nucleotides, an amino acid change, a nomenclature associated with a sequence of nucleotides, a gene symbol, or a molecular biomarker.

9. The method of claim 1, wherein the first genomic type or the second genomic type is a type of alteration, a molecular function, or a nucleotide location within a sequence of nucleotides.

10. The method of claim 1, wherein the first genomic type or the second genomic type is a type of alteration, and wherein the type of alteration is a single-nucleotide polymorphism, multiple-nucleotide polymorphism, insertion, deletion, duplication, mutation, frame shift, repeat expansion, fusion, methylation, or copy number variation.

11. The method of claim 1, wherein the first genomic type or the second genomic type is a molecular function, and wherein the molecular function is a loss of function or a gain of function.

12. The method of claim 1, wherein the alteration nomenclature to be matched is HGVS, DNA alteration, RNA alteration, protein coding variant, MSI, HRD, upregulation of a gene pathway, downregulation of a gene pathway, presence of a protein, absence of a protein, methylation, an epigenetic alteration, or a chromosomal modification.

13. The method of claim 1, wherein the hierarchical rule set includes a heuristic in which the first published medium is ranked higher than the second published medium when the first published medium includes a larger number of alteration nomenclature matches to the subject genomic data than the second published medium.

14. The method of claim 13, wherein the alteration nomenclature matches to the subject genomic data include a match in the first published medium to a pathway that includes a variant identified in the subject genomic data.

15. The method of claim 13, wherein one of the alteration nomenclature matches in the first published medium indicates a resistance to the treatment identified in the second published medium.

16. The method of claim 13, wherein a combination of the alteration nomenclature matches in the first published medium correspond to a different treatment than the treatment identified in the second published medium.

17. The method of claim 1, wherein the one or more evidence metrics include a comparative analysis of an efficacy or of side effects of each treatment identified in each published medium.

18. The method of claim 1, wherein the one or more evidence metrics characterizes a level of evidence published in each published medium.

19. The method of claim 18, wherein characterizing a level of evidence includes a factor quantifying an authoritativeness of a source of each of the first published medium and the second published medium.

20. The method of claim 18, wherein characterizing a level of evidence further comprises determining whether an identified treatment is recognized within the National Comprehensive Cancer Network and, if so, attributing greater weight to a published medium containing that identified treatment.

21. The method of claim 18, wherein characterizing a level of evidence further comprises determining whether an identified treatment was administered within a clinical trial having more than 1000 patients and, if so, attributing greater weight to a published medium containing that identified treatment.

22. The method of claim 1, wherein the one or more evidence metrics includes an identification of whether the treatment identified in a published medium is FDA approved and available to the subject.

23. The method of claim 1, wherein the one or more evidence metrics include a similarity match between a subject disease state and disease states identified in the first and second published media.

24. The method of claim 23, wherein the similarity match includes identifying which of the identified disease states from the first and second published media are closest in semantic meaning to the subject disease state within a disease state ontology and assigning a score based at least in part on a difference in the semantic meaning.

25. The method of claim 23, wherein the similarity match includes identifying which of the identified disease states from the first and second published media relate to a closer organ within a disease state ontology to the subject disease state.

26. The method of claim 23, wherein the similarity match includes identifying which of the identified disease states from the first and second published media has more genomic similarities to the subject disease state.

27. The method of claim 1, wherein the hierarchical rule set excludes information identified in a published medium when the treatment identified in the medium is specific to the disease state identified in the medium and when the identified disease state does not match the subject disease state.

28. The method of claim 1, wherein the hierarchical rule set excludes information identified in a published medium when the subject genomic data corresponds to a resistance or non-responsiveness of the treatment identified in the medium.

29. The method of claim 1, wherein the hierarchical rule set evaluates the treatments identified in the published media and excludes information identified in at least one medium when the treatment identified in that medium would result in overreporting of the same drugs or class of drugs as those identified in a treatment identified in another published medium.

30. The method of claim 1, wherein the reporting template generates an excluded treatment portion within the report distinct from the portion of the report reporting the first treatment, and wherein excluding reporting of the second treatment comprises placing the second treatment into the excluded treatment portion of the report.
